(19) United States
(12) Patent Application Publication
(10) Pub. No.: US 2026/0224124 A1
(43) Pub. Date: Aug. 6, 2026

(54) SYSTEMS AND METHODS FOR MEASURING CRITICAL CLOSING PRESSURE AND TISSUE PERFUSION PRESSURE

(71) Applicant: The General Hospital Corporation, Boston, MA (US)

(72) Inventors: Aaron D. Aguirre, Boston, MA (US); Anand Chandrasekhar, Boston, MA (US); Raimon Padrós I Valls, Boston, MA (US)

(21) Appl. No.: 19/153,314

(22) PCT Filed: Feb. 1, 2024
(86) PCT No.: PCT/US2024/014111

§ 371 Date: Aug. 1, 2025

Related U.S. Application Data
(60) Provisional application No. 63/483,007, filed on Feb. 2, 2023.

(51) Int. Cl. A61B 5/029; A61B 5/00; A61B 5/0225; A61B 5/0245

(52) U.S. Cl. CPC A61B 5/029; A61B 5/02255; A61B 5/0245; A61B 5/7267; A61B 5/746

(57) ABSTRACT

In some aspects, a method for determining critical closing pressure (Pcrit) of a patient is provided. The method includes accessing physiological patient data that characterizes pressure parameters and surrogate flow data. The physiological patient data characteries at least two time points. The method further includes using a processor to determine Pcrit based on the physiological patient data. The method also includes generating a report based on the determined Pcrit of the patient.

### CROSS-REFERENCE TO RELATED APPLICATIONS

This application is based on, claims priority to, and incorporates herein by reference for all purposes, U.S. Provisional Patent Application No. 63/483,007 filed on Feb. 2, 2023.

### STATEMENT REGARDING FEDERALLY SPONSORED RESEARCH

This invention was made with government support under R01HL144515 awarded by the National Institutes of Health. The government has certain rights in the invention.

### BACKGROUND

Modern healthcare relies upon a broad array of information gathering systems and methods to empower clinicians to accurately diagnose and treat patients. However, providing clinicians with the necessary information and do so with the accuracy and consistency required can be extremely challenging. This can be particularly true when analysis and decision making is time limited and/or can radically change outcomes. In just one example, treatment of circulatory shock in critically-ill patients requires management of blood pressure using invasive monitoring, but uncertainty remains as to optimal individual blood pressure targets. Mean arterial pressure (MAP) is often used as a biomarker to help guide treatment. However, optimal individual pressure goals for patients with various diseases and comorbidities remain uncertain. Critical closing pressure (Pcrit) can provide a more individualized alternative or additional measure of cardiovascular health. Pcrit refers to the arterial pressure when blood flow stops, providing a fundamental measure of vascular tone in response to disease and therapy. However, Pcrit is generally not available in clinical care. Thus, systems and methods are needed to assist clinicians with diagnosing and monitoring patient status, and to guide care or therapy in a more personalized way.

### SUMMARY OF THE DISCLOSURE

The present disclosure addresses the aforementioned drawbacks by providing systems and methods for providing new sources of information for clinicians, such as measuring critical closing pressure (Pcrit). The ability to measure Pcrit in a clinical setting can enable personalized patient care that is unavailable using other sources of information. For example, individual Pcrit measurements can inform the use of mean arterial pressure (MAP) during treatment. In this way, a novel biomarker, so-called “tissue perfusion pressure” (TPP), can be defined, which personalizes measures or thresholds of MAP for each patient based on the patient's Pcrit. Using the described systems and methods, individualized Pcrit and TPP can be measured in a clinical setting, such as an intensive care unit, an operating room, during a clinic visit, during out-patient care, or even on a wearable device outside of a clinic.

In some aspects, a method for determining critical closing pressure (Pcrit) of a patient is provided. The method includes accessing physiological patient data that characterizes pressure parameters and surrogate flow data. The physiological patient data characteries at least two time points. The method further includes using a processor to determine Pcrit based on the physiological patient data. The method also includes generating a report based on the determined Pcrit of the patient.

In other aspects, a system for measuring Pcrit of a patient is provided. The system includes a pressure/flow measurement module that is configured to measure physiological patient data. The physiological patient data characterizes pressure parameters and surrogate flow data at two or more time points. The system also includes a processor that is configured to identify pressure parameters that characterize the two or more time points based on the physiological patient data. The pressure parameters include at least one of mean arterial pressure (MAP), systolic blood pressure (SBP), or diastolic blood pressure (DBP). The processor is further configured to identify surrogate flow data that characterizes the two or more time points based on the physiological patient data. The processor is further configured to map a pressure-flow relationship for the two or more time points and to calculate a measure of Pcrit as a zero-flow intercept of the pressure-flow relationship. The processor is also configured to generate a report based on the measure of Pcrit of the patient.

In other aspects, a system for measuring Pcrit of a patient is provided. The system includes a pressure/flow measurement module that is configured to determine physiological patient data, which characterizes cardiovascular dynamics of two or more cardiac cycles. The system also includes a processor that is configured to access a trained machine learning algorithm. The processor is further configured to apply the trained machine learning algorithm to the physiological patient data to estimate a measure of Pcrit for the patient. The trained machine learning algorithm was trained on training data that includes paired physiological patient data and Pcrit label data for a plurality of subjects.

These are but a few, non-limiting examples of aspects of the present disclosures. Other features, aspects and implementation details will be described hereinafter.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1A shows a schematic example of a system that can be used in accordance with the present disclosure.

FIG. 1B shows another schematic example of a system that can be used in a clinical context in accordance with the present disclosure.

FIG. 2A provides a flowchart laying out steps of an example process that can be used to measure critical closing pressure or tissue perfusion pressure in accordance with the present disclosure.

FIG. 2B provides a flowchart laying out steps of another example process that can be used to measure critical closing pressure or tissue perfusion pressure in accordance with the present disclosure.

FIG. 3 provides a flowchart laying out steps of another example process that can be used to measure critical closing pressure or tissue perfusion pressure using a machine learning algorithm in accordance with the present disclosure.

FIG. 4 provides a flowchart laying out steps of an example process to train a machine learning algorithm to measure critical closing pressure or tissue perfusion pressure that can be used in accordance with the present disclosure.

FIG. 5A illustrates a pressure in systemic circulation in the presence of a Starling resistor.

FIG. 5B illustrates a physiological schematic of the systemic circulation with the Starling resistor effect.

FIG. 5C illustrates a circuit model of the systemic circulation that can be used to mathematically represent the pressure flow relationship in the circulation.

FIG. 5D illustrates a theoretical extrapolation of cardiovascular properties in accordance with the present disclosure.

FIG. 6A shows blood pressure data from an example patient in accordance with the present disclosure.

FIG. 6B shows a frequency spectrum of a pulse pressure waveform from an example patient in accordance with the present disclosure.

FIG. 6C shows a plot of mean arterial pressure vs. pulse pressure*heart rate for an example patient in accordance with the present disclosure.

FIG. 6D shows a plot of mean arterial pressure, pulse pressure, and critical closing pressure over time for an example patient in accordance with the present disclosure.

FIG. 7A shows example data of paired central venous pressure (CVP) and mean arterial pressure plotted with respect to critical closing pressure in accordance with some aspects of the present disclosure.

FIG. 7B shows example data plotting critical closing pressure, mean arterial pressure, and tissue perfusion pressure in accordance with some aspects of the present disclosure.

FIG. 7C shows example data of tissue perfusion pressure plotted against systemic vascular resistance (SVR) in accordance with some aspects of the present disclosure.

FIG. 7D shows example data of tissue perfusion pressure plotted against cardiac output in accordance with some aspects of the present disclosure.

FIG. 7E shows example data of tissue perfusion pressure plotted against vasoactive-inotropic score in accordance with some aspects of the present disclosure.

FIG. 7F shows example data of mortality plotted against vasoactive-inotropic score in accordance with some aspects of the present disclosure.

FIG. 8A shows example data comparing tissue perfusion pressure for patients with short length of hospital stay to those with either long length of stay or death, in accordance with some aspects of the present disclosure.

FIG. 8B shows example data of mortality plotted against tissue perfusion pressure in accordance with some aspects of the present disclosure.

FIG. 8C shows example data of length of hospital stay plotted against tissue perfusion pressure in accordance with some aspects of the present disclosure.

FIG. 8D shows example data of maximum lactate levels plotted against tissue perfusion pressure in accordance with some aspects of the present disclosure.

FIG. 8E shows example data of mortality plotted against tissue perfusion pressure separated by mean arterial pressure groupings in accordance with some aspects of the present disclosure.

FIG. 8F shows example data of length of hospital stay plotted against tissue perfusion pressure separated by mean arterial pressure groupings in accordance with some aspects of the present disclosure.

FIG. 9A shows example analysis of tissue perfusion pressure clusters in accordance with some aspects of the present disclosure.

FIG. 9B shows example trajectory analysis of tissue perfusion pressure clusters with respect to mean arterial pressure in accordance with some aspects of the present disclosure.

FIG. 9C shows example trajectory analysis of tissue perfusion pressure clusters with respect to lactate levels in accordance with some aspects of the present disclosure.

FIG. 9D shows example trajectory analysis of tissue perfusion pressure clusters with respect to cardiac output in accordance with some aspects of the present disclosure.

FIG. 9E shows example data of mortality plotted with respect to tissue perfusion pressure clusters in accordance with some aspects of the present disclosure.

FIG. 9F shows example data of reoperation rate plotted with respect to tissue perfusion pressure clusters in accordance with some aspects of the present disclosure.

FIG. 9G shows example data of prolonged ventilation with respect to tissue perfusion pressure clusters in accordance with some aspects of the present disclosure.

FIG. 9H shows example data of length of hospital stay with respect to tissue perfusion pressure clusters in accordance with some aspects of the present disclosure.

FIG. 10A shows example data of mean arterial pressure plotted over time for an individual subject in accordance with some aspects of the present disclosure.

FIG. 10B shows example data of pulse pressure*heart rate plotted over time for an individual subject in accordance with some aspects of the present disclosure.

FIG. 10C shows example data of critical closing pressure plotted over time for an individual subject in accordance with some aspects of the present disclosure.

FIG. 10D shows example data of tissue perfusion pressure plotted over time for an individual subject in accordance with some aspects of the present disclosure.

FIG. 10E shows example data of fluid input/fluid output plotted over time for an individual subject in accordance with some aspects of the present disclosure.

FIG. 10F shows example data of lactate levels plotted over time for an individual subject in accordance with some aspects of the present disclosure.

FIG. 10G shows example data of vasoactive-inotropic score plotted over time for an individual subject in accordance with some aspects of the present disclosure.

FIG. 11A shows an example neural network that may be implemented in accordance with some aspects of the present disclosure.

FIG. 11B shows example results of the neural network of FIG. 11A.

FIG. 12 is a block diagram of an example cardiovascular measurement system that can implement the methods of the present disclosure.

FIG. 13 is a block diagram of example components that can implement the system of FIG. 12.

### DETAILED DESCRIPTION

Before any aspects of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Some implementations of the disclosure are described herein with reference to the accompanying figures. The description, together with the figure, makes apparent to a person having ordinary skill in the art how some implementations of the disclosure may be practiced. The figures are for the purpose of illustrative discussion and no attempt is made to show structural details of an implementation in more detail than is necessary for a fundamental understanding of the teachings of the disclosure. Any drawings herein are not shown to scale. Where dimensions are given in the text or figures, these dimensions provide example values which could be used with one or more example implementations and do not limit the scope or spirit of the disclosed subject matter.

Circulatory shock is one of the most common reasons for admission to an intensive care unit (ICU) and results from inadequate blood pressure and blood flow to support organ function. Causes of circulatory shock include heart failure, overwhelming infection or sepsis, and hemorrhage. Prompt treatment is required to reverse the cause and to restore adequate blood pressure to prevent severe organ injury and death. Consensus guidelines for treatment of shock provide general targets for mean arterial pressure (MAP) that can be used to adjust medications, but optimal individual pressure goals for patients with various diseases and comorbidities remain uncertain. This has been the subject of much study, with prospective and randomized clinical trials looking at different patient populations failing to show a mortality benefit for higher versus lower MAP goals. Results of these studies highlight that MAP alone may be an inadequate single measure of tissue perfusion and new approaches are needed to guide clinical care.

The pressure drop across the circulation depends upon both the inflow arterial pressure (MAP) and the outflow pressure, which is conventionally taken as central venous pressure (CVP). The systemic circulation has a critical closing pressure (Pcrit), which is the arterial pressure when blood flow stops and the circulation collapses. Pcrit can provide a fundamental measure of vascular tone in response to disease or therapy. Thus, the actual perfusion pressure driving flow can be measured as the difference between MAP and Pcrit. Critical closing pressure has been measured in careful animal experiments and in controlled clinical situations such as cardiac surgery where the circulation has been stopped and flow goes to zero. It has not been possible, however, to reliably measure Pcrit in patients with an intact circulation or in routine clinical care.

The present disclosure provides systems and methods that can, for example, be used to measure critical closing pressure in the systemic circulation. Such measurements can, optionally, be acquired continuously. Advantageously, the present disclosure provides the ability to measure Pcrit using non-invasive data, such as blood pressure measured using readily available blood pressure monitors or other non-invasive surrogate measures of flow.

The present disclosure also describes a tissue perfusion pressure (TPP), which may be defined as the difference between mean arterial pressure and critical closing pressure. The TPP can provide unique information compared to other hemodynamic parameters. The examples provided show that TPP can be used to predict risk of mortality, length of hospital stay, and peak blood lactate levels. These results indicate that tissue perfusion pressure may provide an additional target for blood pressure optimization in patients with circulatory shock or other conditions.

Advantageously, the disclosed systems and methods can utilize any data that represents the pressure-flow relationship of the vascular system. In one implementation, for example, direct measurement of pressure (e.g., MAP) can be measured with respect to a direct measurement of flow (e.g., cardiac output). However, since beat-by-beat measures of cardiac output are not readily available outside of select clinical scenarios, surrogate flow data can alternatively be used. As one non-limiting example, pulse pressure (PP)*heart rate (HR) can be used as a surrogate for flow. Such use of PP*HR as a surrogate is based on the relationship between PP and stroke volume (SV), which describes the amount of blood that the heart pumps with each beat. In general, the cardiac output can be described as CO=SV*HR∝PP*HR. Similarly, other data that tracks or is proportional to cardiac output or blood flow can be used in place of PP*HR. Other example surrogate flow data will be described herein.

In general, the pressure-flow relationship can be determined with at least two measurements at varying physiological states. For example, the pressure-flow relationship can be characterized based on measurements of the physiological system at two different flow levels or flow states. However, more data points over a varying flow-level can also be used to increase the robustness of the estimation of Pcrit or TPP. In some implementations, the described system and methods can rely on natural variability of blood flow. As one non-limiting example, the pressure-flow relationship can be measured at various stages (two or more) of the respiratory cycle, which provides natural variation in blood flow. The use of natural flow variability advantageously allows measurement of Pcrit or TPP without requiring external perturbation of the system. In this way, unsafe flow modulation (e.g., stopping a patient's heart or artificially slowing blood flow) is not required. In some implementations, flow can be externally modulated (e.g., by administering a bolus of fluid, performing a maneuver to modulate cardiac output, increasing heart rate, and so forth).

In some implementations, a time-series signal for pulsatile blood pressure (e.g., arterial blood pressure waveform) can be used to characterize the pressure-flow relationship. In a provided example, the pulse pressure (PP) multiplied by the heart rate (HR), PP*HR, has been demonstrated as a surrogate for cardiac output or blood flow. PP*HR can be plotted beat by beat against the mean arterial pressure (MAP) to demonstrate the relative relationship between flow and pressure in the circulation. Periods of linear variation in the MAP vs. PP*HR relationship correspond to periods where the vascular resistance is constant or nearly constant. The “zero-flow” intercept of the fit line for MAP vs. PP*HR can be determined as the pressure when the fit line reaches PP*HR=0. Such intercept can be used to determine the critical closing pressure (Pcrit). Advantageously, this approach does not require separate pressure and flow measurements. As devices do not commonly exist that can provide simultaneous pressure and flow measurements, the disclosed systems and methods have particular importance and flexibility in clinical practice.

In other implementations, other parameters may be used to characterize the pressure of the pressure-flow relationship. For example, systolic blood pressure (SBP) or diastolic blood pressure (DBP) may also be plotted against PP*HR to determine a zero-flow intercept representing Pcrit. Similarly, other surrogates or direct measures for cardiac output or blood flow, including but not limited to a Doppler flow measurement of an artery, may be plotted against blood pressure over multiple flow conditions to determine a pressure-flow relationship and extrapolate to zero-flow to determine Pcrit. In this way, the present disclosure provides flexible methods for measuring and monitoring Pcrit, which can use several types of pressure- and flow-related data, depending on what is available or conveniently measured in the particular clinical or non-clinical setting.

Moreover, enabling the measurement of Pcrit also allows for the calculation of a second parameter, so-called “tissue perfusion pressure” (TPP). As will be described, Pcrit and TPP may be used together. However, no particular examples described below are limiting. These or other measurements or metrics may be utilized together or separately, or any a variety of combinations. TPP is a sensitive measurement of the adequacy of perfusion for a patient. TPP correlates with other metrics of perfusion, including blood lactate, and the level of TPP predicts important outcomes including mortality and length of hospital stay. Pcrit itself is also a fundamental measurement of vascular tone and provides unique information to other available hemodynamic parameters.

Pcrit and TPP can provide new metrics for therapeutic decision making and can be integrated into devices that provide diagnosis or treatment recommendation. For example, in the inpatient hospital setting, Pcrit and TPP can be particularly useful in diagnosing and managing conditions of circulatory shock, acute heart failure, fluid overload, hypertension, and others. In the outpatient hospital setting, these parameters can also be useful in the management of conditions such as heart failure, fluid overload, hypertension, and so forth. Pcrit and TPP can also be useful in profiling the response of the cardiovascular system (e.g., to exercise), similar to the way blood pressure and heart rate response are used to measure response during cardiac stress tests and cardiopulmonary exercise tests used to diagnose disease or to evaluate performance (e.g., as in athletes).

Pcrit and TPP can provide additional blood pressure targets beyond systolic (SBP), diastolic (DBP), and mean (MAP) arterial pressure targets. As one example, Pcrit and TPP can be used to individualize or optimize the selected MAP target for therapeutics.

In some implementations, the disclosed systems and methods allow Pcrit and TPP to be calculated continuously, at high time resolution, which enables rapid diagnosis and rapid adjustment of therapies in dynamic and dangerous clinical situations such as hypotension, shock, and other conditions leading to clinical deterioration and even cardiac arrest. Time-series measurement of Pcrit and TPP can be incorporated into open or closed loop guidance systems for clinicians. On the diagnostic side, these systems may provide early warning or alerting of clinicians to new clinical diagnoses or impending clinical deterioration. From the therapeutic standpoint, defined targets for Pcrit and TPP may be used to optimize therapeutics. An open loop controller may involve presenting a value and a recommendation to a clinician. For example, the value may be directly displayed on a monitor or sent as a push alert to a device carried by the clinician (e.g., phone or pager). The clinician can then use this information to inform therapy decisions. A closed loop controller can tie therapies directly to the measured level of Pcrit or TPP. As one non-limiting example, a drop in TPP may lead to closed-loop adjustment of a vasoactive medication infusing continuously through an intravenous line.

In some implementations, Pcrit or TPP can be calculated in an organ-specific manner. For example, Pcrit can be calculated for the kidney, liver, brain, heart, or other organs by recording and analyzing pressure and flow-related measurements from the arteries selectively supplying such organ. Organ-specific Pcrit measurements can allow for profiling of differential effects of various diseases, such as hypertension or circulatory shock, on individual organ beds. Thus, organ-specific Pcrit and TPP can provide unique biomarkers for disease or end organ injury, similar to how biomarkers like glomerular filtration rate, blood creatinine level, or liver transaminase level are clinically used to identify end organ injury. As Pcrit is a unique measure of vascular tone, it may vary with disease state, with evolution of the disease, and with therapeutic intervention.

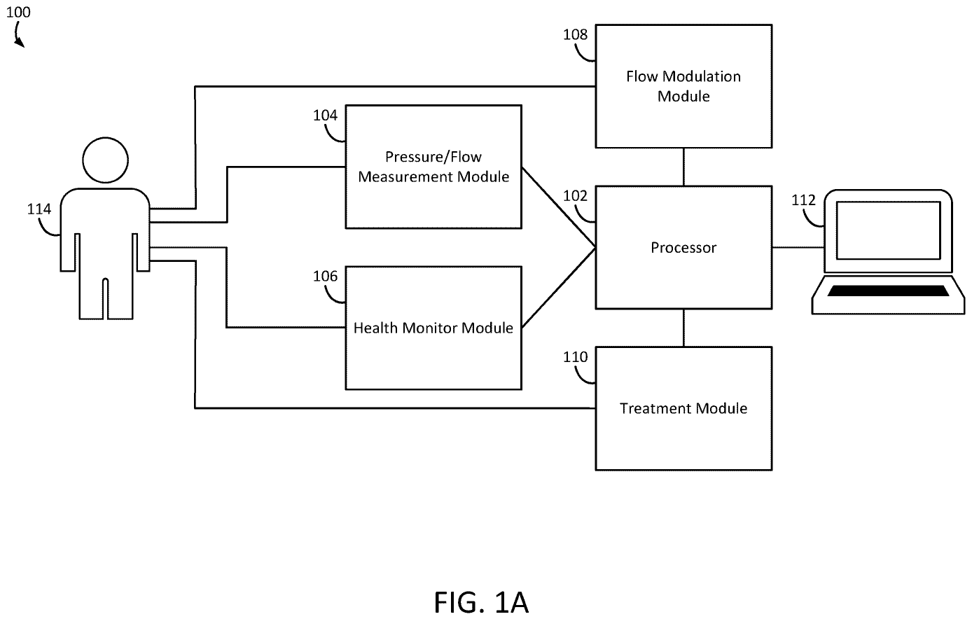

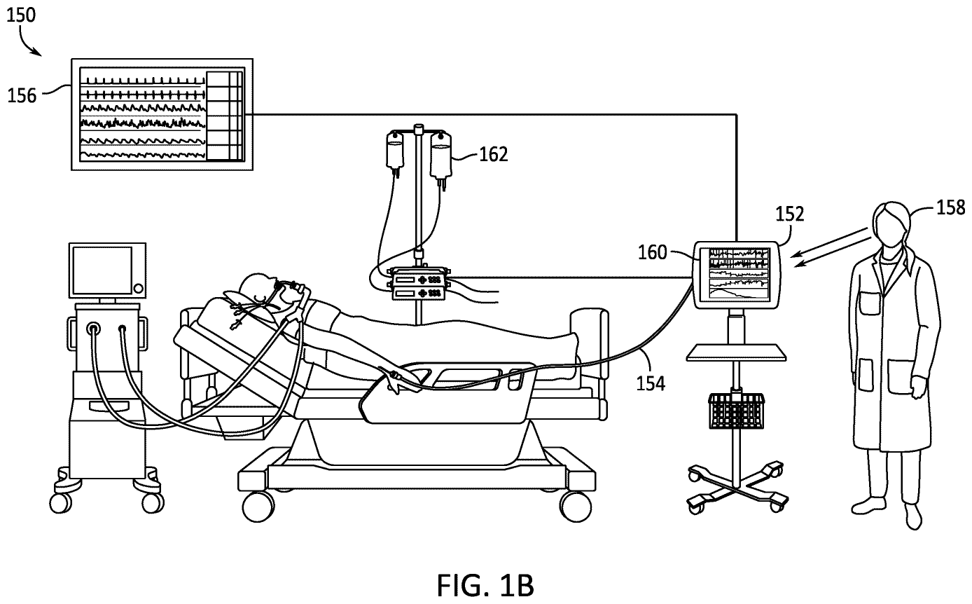

Referring now to FIG. 1A, a schematic system 100 is presented that may be used to measure Pcrit or TPP. The system 100 may include a processor 102 that can control several modules, including a pressure/flow measurement module 104. The modules may further optionally include a health monitor module 106, a flow modulation module 108, and a treatment module 110. The system 100 may also include a user interface 112 that can display measurements to a user or receive input from a user. The various modules (e.g., 104, 106, 108, 110) and the user interface 112 may be connected to the processor 102 by wired or wireless (e.g., Wi-Fi, Bluetooth, and so on) connection. In some implementations some of the various modules may be combined into a single module. For example, the pressure/flow measurement module 104 may be combined with the health monitor module 106 or the treatment module 110 may be used as a flow modulation module 108.

The various modules (e.g., 104, 106, 108, 110) may be in communication with a patient or subject 114 in order to measure or modulate a physiologic parameter of the patient 114. In this way, the pressure/flow measurement module 104, health monitor module 106, or a combination thereof can be used to measure physiological patient data. The physiological patient data can include data that characterizes pressure or flow at two or more time points that are characterized by two physiological states of the system. For example, the physiological patient data can include characterization of pressure and flow of at least two distinct flow states of the system. As described herein, such distinct states can be realized by natural physiological variation or by external flow modulation.

Such physiological patient data may include blood pressure data (e.g., a blood pressure waveform, or discrete measurements of systolic and diastolic blood pressure), blood flow or cardiac output data, surrogate blood-flow data, or patient demographic data. In general, the physiological patient data can be used to derive pressure parameters and surrogate flow data. As will be described in further detail below, the physiological patient data may include or characterize information about the patient's blood pressure (e.g., mean arterial pressure (MAP), systolic blood pressure (SBP), diastolic blood pressure (DBP), pulse pressure (PP)), heart rate (HR), blood flow, or cardiac output at various time points (e.g., over at least two cardiac cycles, at several points throughout the respiratory cycle, before and after a change in flow, and so forth).

In some implementations, the physiological patient data may characterize a patient's blood flow. Such data may be referred to as blood-flow data, surrogate blood-flow data, or surrogate flow data. In general, surrogate flow data can include several different types of data that are related to blood flow. For example, surrogate blood flow data can include direct measurements of blood flow (e.g., using Doppler flow measurements). Surrogate blood flow data may also include physiological measurements that are proportional to absolute flow data. As non-limiting examples, the surrogate blood-flow data may include blood pressure data, heart rate data, photoplethysmography (PPG) data, electrocardiogram data (ECG), echocardiogram data, flow data (e.g., Doppler flow data, 4D flow magnetic resonance imaging data), medical imaging data (e.g., ultrasound images, 4D flow magnetic resonance imaging data), cardiac imaging data, cardiovascular imaging data, optical spectroscopy data, arterial tonometry data, oxygen saturation data (O₂), and so forth. In one example, PP*HR can be used as surrogate flow data.

The processor 102 can process physiological patient data, which may be measured or received by the pressure/flow measurement module 104, as will be described in further detail below. For example, the processor 102 can calculate mean arterial pressure (MAP), heart rate, systolic blood pressure, diastolic blood pressure, pulse pressure, or other parameters using the physiological patient data. The processor 102 may also process (e.g., filter) the physiological patient data or calculated parameters. The processor 102 may also store and use an algorithm to calculate Pcrit and TPP based on the pressure data or other physiological patient data. The processor 102 can also be used to calculate or set pressure thresholds. For example, the processor 102 can calculate an individualized MAP or TPP threshold for administering treatment or medication to the patient 114.

The processor may also communicate with the user interface 112 to generate a report, display the measured or calculated parameters, alert healthcare providers of critical biomarkers, record and display parameters of past administration of treatment or flow modulation, and so on. In this way, the user interface may be used to display any relevant or desired parameters of the modules (e.g., 104, 106, 108, 110) or calculated parameters. The user interface 112 can also receive user inputs, such as instructions for drug administration, adjustment of pressure thresholds, patient demographics, desired timing of pressure or flow measurements, and so forth.

In some implementations, the pressure/flow measurement module 104 can be used to measure systolic blood pressure, diastolic blood pressure, pulse pressure, heart rate, MAP or other relevant flow- or pressure-related metrics. As a non-limiting example, the pressure/flow measurement module 104 may include a blood pressure monitoring device, such as an indwelling arterial pressure catheter (e.g., fluid-filled catheter or solid-state transducer). For example, the blood pressure monitor may include a catheter placed in an artery (e.g., radial artery, brachial artery, femoral artery, pulmonary artery, aorta etc.) of the patient 114. Such catheter may also be referred to as an arterial line. In this way, the pressure/flow measurement module 104 can be used to measure arterial blood pressure at two or more time points or to measure an arterial blood pressure waveform over time.

The blood pressure monitoring device may also include a non-invasive device, such as a sphygmomanometer or blood pressure cuff. As one non-limiting example, the non-invasive pressure monitoring device may include a blood pressure cuff that provides a standard oscillometric (e.g., inflate/deflate) blood pressure measurement and can hold a static cuff pressure and measure a pulsatile pressure waveform. Other non-limiting examples of non-invasive devices include volume-clamp pressure devices (e.g., commercially-available finger cuff devices, FlowTrac™ available from Edwards Lifesciences Corporation) or ring-based sensors. For example, the blood pressure monitor may also include a blood pressure cuff placed around an arm or finger of the patient 114. In this way, the pressure/flow measurement module 104 can be used to measure blood pressure at various time points (e.g., during two or more cardiac cycles).

In some implementations, the pressure/flow measurement module 104 may include other patient monitors or measurement devices that can be used to characterize pressure- or flow-related parameters. For example, the pressure/flow measurement module may include an electrocardiogram (ECG or EKG) measurement device, a photoplethysmogram (PPG) measurement device, an O₂ saturation monitor, a medical imaging device (e.g., ultrasound, MRI), an optical spectroscopy device, an arterial tonometry device, or another cardiovascular sensing or imaging device configured to measure cardiac-related parameters. As will be described in further detail below, such physiological patient data (e.g., ECG, PPG, O₂ saturation, optical spectroscopy, arterial tonometry, 4D flow images, echocardiogram, cardiovascular sensing data, or cardiovascular imaging data) may be used to estimate a blood pressure waveform or directly estimate Pcrit or TPP. Such data may be used to explicitly extract blood pressure data without directly measuring blood pressure.

As a non-limiting example, an ECG signal can be used to extract blood pressure parameters, which may include processing the ECG signal with a machine learning algorithm. In some implementations, the ECG signal can be calibrated with blood pressure data in order to extract pressure parameters (e.g., MAP, SBP, DBP, HR, PP) from the ECG signal. As another non-limiting example, the pressure/flow measurement module 104 may include a photoplethysmography (PPG) device, which may include an infrared light source and optical sensor. Such PPG signal can be processed to extract blood pressure parameters (e.g., MAP, SBP, DBP, HR, PP), using a calibration or a machine learning algorithm, for example.

As another non-limiting example, the pressure/flow measurement module 104 may include a cardiovascular sensing or imaging device, such as an echocardiography device, a magnetic resonance imaging system, or an arterial Doppler measurement device. The cardiovascular imaging or sensing data can be used to extract pressure- and flow-related parameters. As one non-limiting example, the Doppler ultrasound data (e.g., flow velocity) can be used to characterize blood flow, which can serve as surrogate flow data. As another non-limiting example, imaging data can be used to determine stroke volume, which can serve as surrogate flow data. The cardiovascular sensing data or imaging data can also be used with a machine learning algorithm or other algorithm to explicitly extract pressure and flow parameters. In other implementations, the cardiovascular sensing data or imaging data can be used with a machine learning algorithm to directly estimate Pcrit or TPP.

In some implementations, the pressure/flow measurement module 104 may include several monitors that simultaneously or alternatingly measure blood pressure at various anatomical locations. For example, the pressure/flow measurement module 104 may include a blood pressure cuff on both right and left arms or arterial lines in the radial and brachial arteries. As another example, the pressure/flow measurement module 104 may include a non-invasive blood pressure cuff and a non-invasive PPG monitor.

The health monitor module 106 may provide auxiliary or complementary health data. For example, the health monitor module 106 may include a heart rate or pulse monitor. The health monitor module 106 may also provide discrete or continuous measurement of other relevant parameters, such as concentrations of blood biomarkers (e.g., oxygen), respiration rate, body temperature, and others. The health monitor module 106 may provide additional information that may be used in open- or closed-loop treatment decisions.

The pressure/flow measurement module 104, health monitor module 106, or a combination thereof can provide a patient monitor and may be referred to as such. In some implementations, the health monitor module 106 may be combined or partially combined with the pressure/flow measurement module 104. For example, blood biomarkers may be measured by sampling blood via an arterial line of the pressure/flow measurement module 104. In some implementations, the pressure/flow measurement module 104, the health monitor module 106, or both may be provided to the patient 114 in the form a wearable device (e.g., smart watch, smart phone, PPG from a finger sensor or a wearable body patch, a direct pressure sensor on the skin overlying an artery, or similar).

The system 100 may also optionally include a flow modulation module 108. The flow modulation module 108 can be used to modulate the physiological system of the patient 114 in order to perturb the patient's blood flow or a parameter of the patient's blood flow (e.g., stroke volume, cardiac output, or local arterial blood flow). For example, the flow modulation module 108 may be used to administer a drug or a bolus of fluid (e.g., saline) into the blood stream of the patient 114 to modulate the patient's cardiac output or heart rate. In this way, blood pressure and surrogate flow data can be measured by the pressure/flow measurement module 104 before and after the modulation is achieved. In other implementations, the flow modulation module 108 may be omitted, and the system measurement can rely on natural variation in cardiac output, which leads to natural variation in blood pressure. For example, blood pressure can be measured at various stages of the respiratory cycle, which typically causes variation in stroke volume and cardiac output with variations in filling (preload) of the heart.

The treatment module 110 can optionally be used to provide open- or closed-loop treatment or treatment titration for the patient 114. For example, the treatment module 110 may administer drugs (inotropic, vasoactive, or chronotropic medications), medicament, or other treatment (e.g., fluid administration, recommendation for use of advanced mechanical circulatory support) to the patient based on feedback from the processor 102. Such feedback may be informed by a user input via the user interface 112 or by pressure measurements and thresholds generated by the processor 102. In this way, the processor can control the treatment module to administer a desired treatment to the patient 114.

FIG. 1B shows a schematic of an example system 150 that may be used to measure Pcrit or TPP. While system 150 is shown in the context of an intensive care unit (ICU) setting, similar systems may be used in other clinical settings, such as outpatient settings, clinic or urgent care visits, operating rooms, and so forth. Advantageously, the system 150 can be used for a patient or subject with an intact circulatory system, allowing for convenient, safe, and continuous monitoring of Pcrit or TPP.

As FIG. 1B shows, a processor 152 can communicate with a blood pressure monitor 154 (e.g., arterial catheter or noninvasive blood pressure monitor). The system 150 can integrate existing monitoring signals by providing communication between the processor and external monitors 156. The processor 152 can display measured and calculated parameters to a healthcare professional or other user 158 using a user interface or display 160. Such display 160 can inform the user 158 of risk stratification, individualized blood pressure targets, medication titration, fluid administration, and so forth. The processor 152 can also control a medication titration system 162 based on the measured or calculated pressure metrics, such as MAP, Pcrit, TPP, and so on.

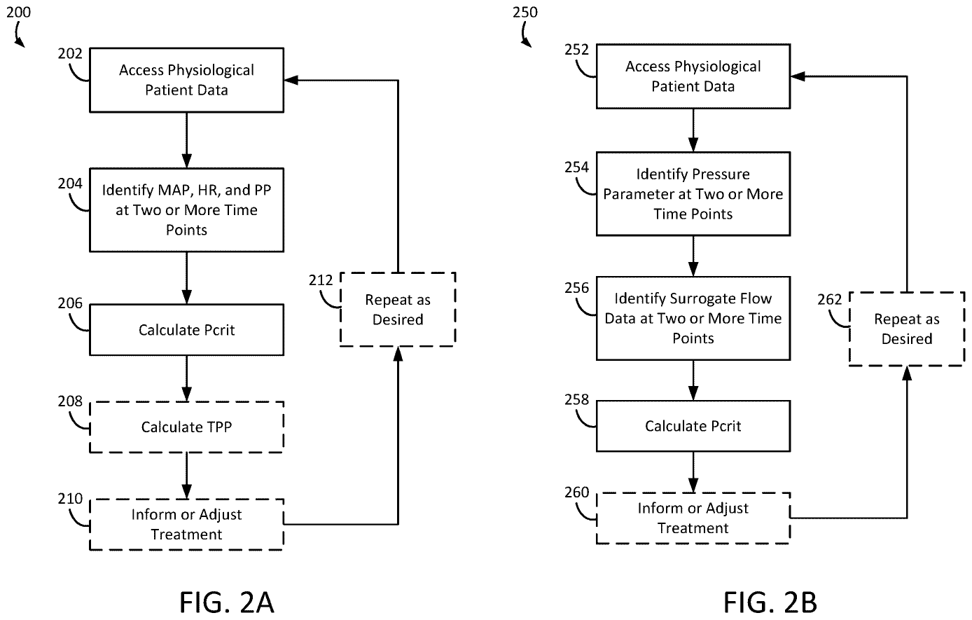

FIG. 2A provides a flowchart of a process 200 that can be used to measure Pcrit or TPP for a patient or subject. In some implementations, process 200 can be performed by system 100 or similar. For instance, process 200 can be performed using invasive or non-invasive physiological patient data.

The process 200 includes accessing physiological patient data, as indicated in block 202. Such physiological patient data may characterize cardiovascular dynamics. For example, the physiological patient data may include data from which MAP, HR, and PP can be derived. In this way, the physiological patient data may be used to implicitly or explicitly characterize MAP, SBP, DBP, HR, or PP.

Accessing physiological patient data may include using a computer system to access stored data. Accessing physiological patient data may also include measuring a blood pressure waveform, using an invasive arterial catheter, for example. As a non-limiting example, the blood pressure waveform may be sampled with a rate of 120 Hz. Such blood pressure waveform may be measured using an arterial catheter or arterial line as previously described.

Accessing physiological patient data may also include non-invasively measuring blood pressure (e.g., systolic and diastolic blood pressure) at two or more time points, such as during two cardiac cycles. For example, blood pressure may be measured using a non-invasive blood pressure cuff before and after a flow modulation or at two points throughout the respiratory cycle. For example, the blood pressure may be measured at an initial time point; the cardiac output can be modulated (e.g., by administering a bolus of fluid or drug or by natural pressure variation throughout the respiratory cycle); then the blood pressure can be measured at a second time point. Such use of external blood flow modulation may be preferable when using a non-invasive monitor of blood pressure. While the use of two time points may be sufficient, blood pressure may be measured for several time points (e.g., over the course of external cardiac output modulation, throughout the course of natural variation in stroke volume or cardiac output as occurring over one or more respiratory cycles, or with natural variation in heart rate).

The blood pressure data may include discrete measurements of systolic blood pressure (SBP) and diastolic blood pressure (DBP) at two or more time points (e.g., two or more cardiac cycles). The blood pressure data may also include a measure of heart rate. For example, the heart rate may be nearly instantaneously measured using a heart rate sensor or monitor. The heart rate may also be measured as an average over a time period. For example, a patient or caregiver can measure a patient's heart rate using their fingers (e.g., placing fingers on the patient's neck or wrist and counting heart beats over a given time period, such as one minute).

Accessing physiological patient data may also include non-invasively measuring pressure-related data that can be used to derive MAP, HR, and PP. For example, the physiological patient data may include PPG data, ECG data, O₂ saturation data, optical spectroscopy data, arterial tonometry data, or cardiovascular imaging data.

As indicated in block 204, the physiological patient data can be processed in order to identify pressure- or flow-related parameters. Such parameters may include MAP, HR, and PP at two or more time points. As a non-limiting example, a blood pressure waveform can be used to identify MAP, HR, and PP. The maximum and minimum locations along the blood pressure waveform can be identified to calculate MAP, HR, and PP. In some implementations, overall minima and maxima can be used as initial estimates to locate maximum and minimum for each cardiac beat. For example, a window of 100 ms can be centered on the initial estimate to assess the unfiltered waveform to determine maximum and minimum locations. A cardiac cycle can be defined between two adjacent minima. The height and time average of the blood pressure waveform within one cardiac cycle can be used as the PP and MAP, respectively. The HR can be identified based on the time-width of the cardiac cycle (ΔTw) as HR=60/ΔTw.

In some implementations, the physiological patient data can include blood pressure data measured at two time points. This blood pressure data may characterize MAP, HR, and PP or be used to explicitly derive or estimate MAP, HR, and PP. As one non-limiting example, MAP can be estimated as

$$MAP = \frac{1}{3} S B P + \frac{2}{3} DBP$$

and pulse pressure can be estimated as PP=SBP−DBP.

In some implementations, the physiological patient data includes pressure- or flow-related data, such as PPG or ECG data. Such non-invasive data can be used to derive MAP, HR, and PP in block 204. For example, PPG data can be previously calibrated to blood pressure waveform data. The calibration can be applied to newly acquired PPG data in order to estimate MAP, HR, and PP. Such calibration may also include a discrete measurement of blood pressure provided by a non-invasive blood pressure monitor.

In some implementations, block 204 may also include filtering the data. For example, the raw blood pressure waveform may be filtered to remove outliers or noise. As one example, the blood pressure data can be filtered with a low-pass filter to remove high-frequency signals. In some implementations, the calculated parameters can be filtered to remove outliers. For example, outlier MAP, HR, or PP measurements can be discarded and remeasured if outside a desired threshold (e.g., 5%-95% of all measurements). As another example, intermediate parameters (e.g., PP×HR) can be filtered to remove outliers (e.g., outside 5%-95% of all measurements).

The MAP, PP, and HR can be used in block 206 to calculate Pcrit. For example, the MAP and the product of PP and HR (PP×HR) can be plotted for all acquired time points (e.g., two or more, all non-outliers over one minute period). A line can be fit to the MAP vs. PP×HR curve to calculate a pressure-axis intercept. This intercept can be used as an estimate of Pcrit. In this way, the intercept (PP*HR=0) represents the blood pressure of the zero-flow state, as extrapolated from the mapped pressure-flow relationship.

Calculating Pcrit may optionally include filtering of Pcrit estimations. For example, estimated Pcrit values can be discarded if the slope or pressure-axis intercept of the best-fit line are negative or below another predetermined threshold. As another example, Pcrit values can be discarded if the coefficient of determination (r²) of the fit is below a given threshold (e.g., 0.3).

In some implementations, Pcrit can be used to better interpret a MAP measurement for a given subject. For example, Pcrit can be used to set an individualized MAP threshold for treating a patient. Thus, Pcrit can be interpreted with or without other measured parameters of patient data in block 210 to inform or adjust patient treatment (e.g., whether to administer a drug or other treatment, what drug dose to use, whether to discharge or continue monitoring a patient, and so forth).

An additional parameter, called tissue perfusion pressure (TPP) can optionally be calculated, as indicated in block 208. TPP may help to interpret Pcrit and MAP together in a more intuitive way, consistent with clinical practice. TPP can be calculated as the difference between the average MAP over a time interval (e.g., 1 minute or two time points) and the estimated Pcrit. Block 208 may optionally include filtering Pcrit, MAP, or calculated TPP values as desired to improve data quality. The TPP can optionally be used in block 210 to inform clinical care decisions. For example, treatment may be administered if the TPP is outside a desired threshold.

Block 210 may include generating a report that can be stored or provided to a processor or caretaker. For example, the report may include a measure of Pcrit or TPP. The report may also include a measure of the confidence of the Pcrit or TPP estimate (e.g., r², number of outliers). The report may also include a timeseries of Pcrit or TPP measurements or other measured parameters (e.g., MAP, HR, PP, blood pressure, PPG data, ECG data, O₂ saturation data). The report may also include a record of treatments administered to the patient. The report may also include a treatment recommendation that is based on the measure of Pcrit, MAP, TPP, or a combination thereof.

Block 210 can also include displaying the measured or calculated parameters on a user display to provide clinical information to caregivers or another user. In this way, process 200 can provide an open feedback loop in which treatment can be adjusted by a clinician in nearly real time. For example, the treatment can be maintained or adjusted with each measurement of Pcrit at a frequency of the measurement stride or repetition time (e.g., one minute). The measured parameters can also be communicated or stored in other ways. For example, an alarm can be used to indicate a measurement of Pcrit or TPP that is above or below a desired threshold. In some implementations, block 210 can provide information to a processor that can automatically adjust treatment. In this way, process 200 can be used to provide closed-loop control of patient care. Such closed-loop control can be continuous with the frequency of the Pcrit or TPP measurement (e.g., 1 minute).

The pipeline can be repeated as indicated in block 212. For example, Pcrit or TPP can be calculated over time to closely monitor patient health. As a non-limiting example, TPP can be calculated using a sliding 1 minute window while a patient is in the ICU. As another example, Pcrit or TPP can be measured using a sliding window between 1 s and 1 day (e.g., 1-60 s, 10-60 s 1-10 minutes, 1-60 minutes, 1-24 hours). In this way, treatment can be continuously titrated or adjusted based on patient-specific and recent data.

In some implementations, blood pressure waveform data may not be available. However, other pressure and surrogate flow data can be used to determine Pcrit, as demonstrated in FIG. 2B. FIG. 2B provides a flowchart of a process 250 that can be used to measure Pcrit or TPP. While process 250 is similar to process 200 of FIG. 2A, process 250 provides further flexibility, allowing use of alternative data sources. In some implementations, process 250 can be performed by system 100 or similar. For instance, process 250 can be performed using invasive or non-invasive physiological patient data.

Process 250 includes accessing physiological patient data, as indicated in block 252. Block 252 may include measuring physiological patient data (e.g., using system 100) or accessing stored physiological patient data. In general, the physiological patient data can be used to characterize surrogate flow data and pressure parameters at two or more time points. For example, such physiological patient data may include pressure waveform data, cardiovascular imaging data, cardiac imaging data (e.g., MRI images, 4D flow data, echocardiogram data), cardiac flow data (e.g., Doppler ultrasound flow measurements), cardiac output data, electrocardiogram data, PPG data, 0₂ saturation data, discrete blood pressure measurement data, and so forth.

The physiological patient data can be used in block 254 to identify a pressure parameter at two or more time points. As a non-limiting example, the pressure parameter may be MAP, as previously described. As another non-limiting example, the pressure parameter may be SBP, DBP, or another measure or characterization of blood pressure. Such pressure parameters (e.g., MAP, SBP, DBP, PP, and so forth) may be identified based on blood pressure waveform data, discrete measurements of blood pressure, or otherwise derived from pressure-related data. As a non-limiting example, pressure parameters could be derived from the physiological patient data using a machine learning model or algorithm (e.g., neural network), which was previously trained to relate or calibrate the physiological patient data to pressure parameters. As non-limiting examples of such physiologic patient data, pressure parameters could be derived from PPG signals, ECG signals, arterial tonometry signals, ultrasound imaging, or other cardiovascular imaging data.

Block 256 includes identifying surrogate flow data at the two or more time points. In some implementations, the surrogate flow data can include a direct measure of blood flow. In other implementations the surrogate flow data may be an indirect measure of blood flow. In still other implementations, the surrogate flow data can include a measure of flow-related parameters that are proportional to blood flow. As one non-limiting example, the surrogate flow data can be defined from blood pressure measurements as PP*HR, as previously described (e.g., as in FIG. 2A). As another non-limiting example, the surrogate flow data may include flow measurements or flow velocity identified from Doppler sensing or Doppler ultrasound imaging. As other non-limiting examples, the surrogate flow data may include a measure of stroke volume or ejection fraction identified from medical imaging data, such as echocardiography. As another non-limiting example, the surrogate flow data may include PPG data. As another non-limiting example, the surrogate flow data may include optical measurements that are proportional to blood flow, including near-infrared spectroscopy, diffuse correlation spectroscopy, speckle contrast imaging, or Doppler optical coherence tomography. As another non-limiting example, the surrogate flow data can be defined from blood pressure surrogate measurements such as arterial tonometry by calculating a parameter proportional to PP*HR. In some implementations, identifying surrogate flow data may include processing the physiological patient data using a machine learning algorithm (e.g., neural network) that was previously trained to relate or calibrate the physiological patient data to blood flow data.

In some implementations, the pressure parameters and flow surrogate data can be paired such that each pair of pressure measurement and flow measurement represents a particular state of the cardiovascular system. For example, for each pair, the pressure parameter and flow surrogate data can be measured at the same time point. As another example, for each pair, the pressure parameter and flow surrogate data can be measured for the same or similar physical state (e.g., prior to flow modulation and after flow modulation). Such paired data allows characterization of a pressure-flow relationship.

Blocks 254 and 256 may also include processing the raw physiological patient data. As a non-limiting example processing may include filtering the raw physiological patient data to remove noise or outlier data. In some implementations, processing the data could also include extracting relevant parameters from the raw data. For example, flow velocity can be determined from Doppler flow measurement data. As another non-limiting example, stroke volume can be determined from cardiac imaging data using automated or manual image processing techniques. Other processing may also be applied to determine relevant pressure parameters or surrogate flow data. In some implementations, blocks 254 and 256 can also include filtering of pressure parameters or surrogate flow data. For example, the pressure parameters or surrogate flow data can be filtered to remove noise or outliers.

In general, the paired pressure and surrogate flow data will have variation over the two or more time points. Such physiological variation may be observed based on the natural variation of pressure and flow (e.g., naturally occurring changes over the course of a respiratory cycle) or may be artificially modulated using a flow modulation technique (e.g., applying fluid bolus, administering drugs or treatment to change heart rate, and so forth). This variability of pressure and flow over time (e.g., two or more time points) can be used to characterize a pressure-flow relationship. This pressure-flow relationship can be implicitly characterized in block 258 in order to calculate Pcrit. By mapping paired pressure-flow data for at least two time points or flow states, a zero-flow point can be interpolated, which represents the pressure in a zero-flow condition. This pressure can be referred to as Pcrit. For instance, the paired pressure parameter and surrogate flow data can be fit to a line to identify or estimate Pcrit as the zero-flow intercept. For example, the two or more pressure parameter-surrogate flow pairs can be plotted with pressure parameter on a y-axis and surrogate flow data on an x-axis. The paired data can be fit using linear regression or another fitting method. In this example, the y-intercept can of the fit can be used as an estimate for Pcrit.

As previously described, Pcrit can be used further to calculate TPP or to otherwise inform treatment as indicated in block 260. The process can be repeated as indicated in block 262. For example, the process can be repeated continuously (e.g., every 1 minute) to continually monitor a patient's Pcrit and inform treatment decisions or titrate medication levels. As another example, the process can be repeated longitudinally (e.g., daily, monthly, yearly) to monitor a patient's cardiac health over time.

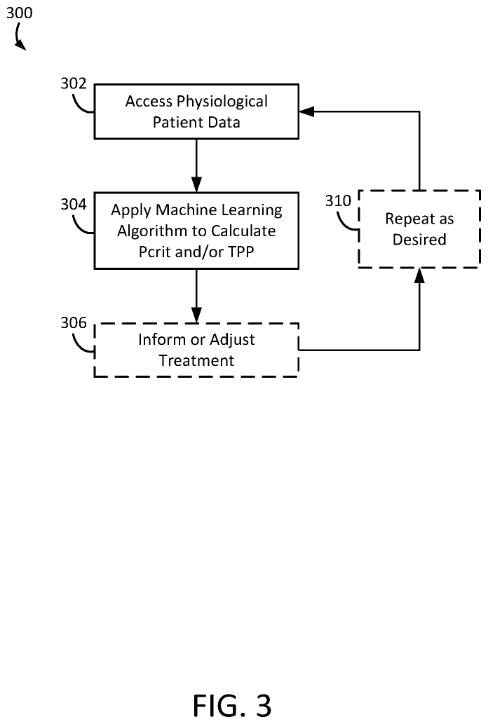

FIG. 3 provides another example process 300 that can be used to calculate Pcrit or TPP. As will be described, the neural network or other machine learning algorithm takes physiological patient data as input data and generates measurements of Pcrit or TPP as output data. In some implementations, the use of a neural network can circumvent the need to explicitly calculate MAP, SBP, DBP, HR, PP, or surrogate flow data.

Physiological patient data can be accessed with a computer system in block 302. Accessing physiological patient data may include accessing data from a suitable storage device or other memory. Accessing the data may also include measuring data from which pressure parameters (e.g., MAP, SBP, or DBP) HR, and surrogate flow data could be derived and transferring or otherwise communicating the data to the computer system. For example, the physiological patient data may include blood pressure data, which may include blood pressure (e.g., SBP and DBP) measured at two or more time points. As another example, the blood pressure data may include an arterial blood pressure waveform measured from an indwelling pressure catheter. The physiological patient data may also include a timeseries of ECG data, PPG data, O₂ saturation data, cardiovascular imaging data, Doppler flow data, echocardiogram data, optical spectroscopy data, arterial tonometry data, or other cardiac- or flow-related data measured over time.

A machine learning algorithm or trained neural network can be accessed by the computer system and applied in block 304. Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed.

In general, the neural network is trained, or has been trained, on training data in order to estimate Pcrit or TPP based on the physiological patient data. The machine learning algorithm may include a neural network architecture (e.g., artificial neural network (ANN), convolutional neural network (CNN), recurrent neural network (RNN), long short-term memory network (LSTM), and so forth), or other supervised learning algorithms (including but not limited to regression, support vector machine, random forest, gradient boosting, and so forth) trained to estimate Pcrit or TPP from time-series data.

An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs. In an example in which the artificial neural network calculates Pcrit or TPP, the output layer may include, for example, a number of different nodes, where each different node corresponds to a different calculated cardiac metric. A first node may indicate Pcrit, and a second node may indicate TPP, for example. In some implementations, a node may be used to estimate a blood pressure waveform from non-invasive physiological patient data. In some implementations, a node may be used to estimate blood pressure parameters from physiological patient data. In some implementations, a node may be used to estimate blood flow data or surrogate flow data from the physiological patient data. Thus, the physiological patient data can be input to the trained neural network in block 304 in order to calculate Pcrit, TPP, or intermediate data (e.g., a blood pressure waveform, pressure parameters, surrogate flow data) from which Pcrit or TPP can be calculated.

Use of a machine learning algorithm may increase computation speeds, which may provide faster real-time data to be available to make fast treatment decisions. Additionally, the use of a machine learning algorithm may reduce the need to explicitly calculate or derive specific parameters, such as MAP, SBP, DBP, HR, PP, and flow from the physiological patient data. This may reduce the propagation of error in the calculation of Pcrit or TPP, for example. The use of a machine learning algorithm may also provide a more accurate or more convenient pipeline for calculating Pcrit or TPP using readily available invasive or non-invasive data (e.g., arterial pressure waveforms, PPG, ECG, blood pressure cuff data, imaging data, and so forth). For example, use of a machine learning algorithm may circumvent the need to explicitly calibrate PPG data with blood pressure waveform data.

As a non-limiting example, the machine learning algorithm may include a CNN using a residual network (ResNet) architecture. The algorithm may process raw arterial blood pressure (ABP) waveform data to compute Pcrit and TPP metrics. As another non-limiting example, the machine learning algorithm may be trained to process non-invasive data, such as PPG, ECG, or continuous cardiovascular sensing or imaging data, to calculate Pcrit or TPP.

In some implementations, block 304 may include using a machine learning algorithm to estimate a blood pressure waveform or pressure parameters from non-invasive data. Such blood pressure waveform or pressure parameters can be used to calculate Pcrit or TPP, as described in the context of FIGS. 2A-2B, for example. In some implementations, block 304 may include using a machine learning algorithm to estimate MAP, SBP, DBP, HR, and PP from non-invasive data. Such parameters can be used to estimate Pcrit or TPP, as previously described. In some implementations, block 304 may include using a machine learning algorithm to also estimate flow surrogate data from physiological patient data. Such flow surrogate data may be used with blood pressure data to estimate Pcrit or TPP, as previously described.

In some implementations, the physiological patient data accessed in block 302 may include a timeseries of non-invasive cardiovascular flow-related data (e.g., PPG or Doppler ultrasound flow measurements) and a measure of blood pressure at one or more time points. The machine learning algorithm can use the dynamic and static data in block 304. In this way, the non-invasive data can be implicitly calibrated using the discrete measure of blood pressure.

The calculated Pcrit or TPP can be stored for future use or used to inform or adjust treatment, as indicated in block 306. Block 306 may also include generating a report that can be relayed to a caretaker or stored for future use. The process can be repeated in block 310 as desired. For example, Pcrit or TPP can be measured before and after treatment is applied. As another example, Pcrit and TPP can be measured continually (e.g., every one minute) to monitor a patient's status and indicate whether drugs intervention is recommended.

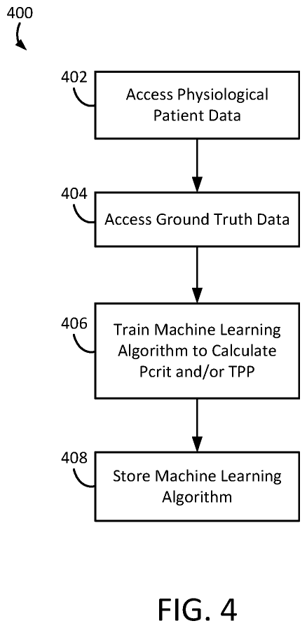

FIG. 4 provides an example process 400 that may be used to train a machine learning algorithm that can be applied to calculate Pcrit or TPP based on physiological patient data. In general, the neural network(s) can implement any number of different neural network architectures. For instance, the neural network(s) could implement a convolutional neural network, a residual neural network, or the like. Alternatively, the neural network(s) could be replaced with other suitable machine learning or artificial intelligence algorithms, such as those based on supervised learning, unsupervised learning, deep learning, ensemble learning, dimensionality reduction, and so on.

The physiological patient data can be accessed in block 402, which may serve as training data. The physiological patient data can be accessed by a computer system. Accessing the data may include retrieving such data from a memory or other suitable data storage device or medium. Alternatively, accessing the training data may include measuring or acquiring such data with a patient monitor (e.g., 104 or 106) and transferring or otherwise communicating the data to the computer system.

As previously described, the physiological patient data may include discrete blood pressure measurements for two or more time points (e.g., cardiac cycles), a blood pressure waveform measured for a period of time (e.g., 1 minute), non-invasive cardiovascular data (e.g., PPG, ECG), or other cardiovascular-related measurements that contain information about a pressure-flow relationship (e.g., ultrasound, Doppler flow, optical or other imaging data). Such physiological patient data can include data for several subjects or patients and be paired to ground truth data, accessed in block 404. In some implementations, Pcrit or TPP values can serve as ground truth data. Thus, such Pcrit or TPP values may be referred to as Pcrit label data and TPP label data, respectively. In other implementations, a blood pressure waveform or pressure parameters can serve as ground truth data, such that the machine learning algorithm can be trained to calibrate the non-invasive physiological patient data with a blood pressure waveform. Thus, the machine learning algorithm can be trained in block 406 to directly calculate Pcrit or TPP or to calculate a blood pressure waveform or pressure parameters from which Pcrit or TPP can be calculated, as previously described. In some implementations, blood flow measurements can serve as ground truth data. In this way, the machine learning algorithm can be trained in block 406 to calculate surrogate flow data from which Pcrit or TPP can be calculated.

One or more neural networks (or other suitable machine learning algorithms) are trained on the training data, as indicated at block 406. In general, the neural network can be trained by optimizing network parameters (e.g., weights, biases, or both) based on minimizing a loss function. As one non-limiting example, the loss function may be a mean squared error loss function.

Training a neural network may include initializing the neural network, such as by computing, estimating, or otherwise selecting initial network parameters (e.g., weights, biases, or both). During training, an artificial neural network receives the inputs for a training example and generates an output using the bias for each node, and the connections between each node and the corresponding weights. For instance, training data can be input to the initialized neural network, generating output as measurements of Pcrit or TPP. The artificial neural network then compares the generated output with the ground truth data of the training example in order to evaluate the quality of the estimation. For instance, the estimated parameters can be passed to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function.

The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The artificial neural network can be constructed or otherwise trained based on training data using one or more different learning techniques, such as supervised learning, unsupervised learning, reinforcement learning, ensemble learning, active learning, transfer learning, or other suitable learning techniques for neural networks. As an example, supervised learning involves presenting a computer system with example inputs and their actual outputs (e.g., categorizations). In these instances, the artificial neural network is configured to learn a general rule or model that maps the inputs to the outputs based on the provided example input-output pairs.

The trained machine learning algorithm can then be stored for later use, as indicated at block 408. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

The following are non-limiting examples.

### Examples

### Theory

### Physiological Model: TPP=MAP−Pcrit is a Measure of Tissue Perfusion

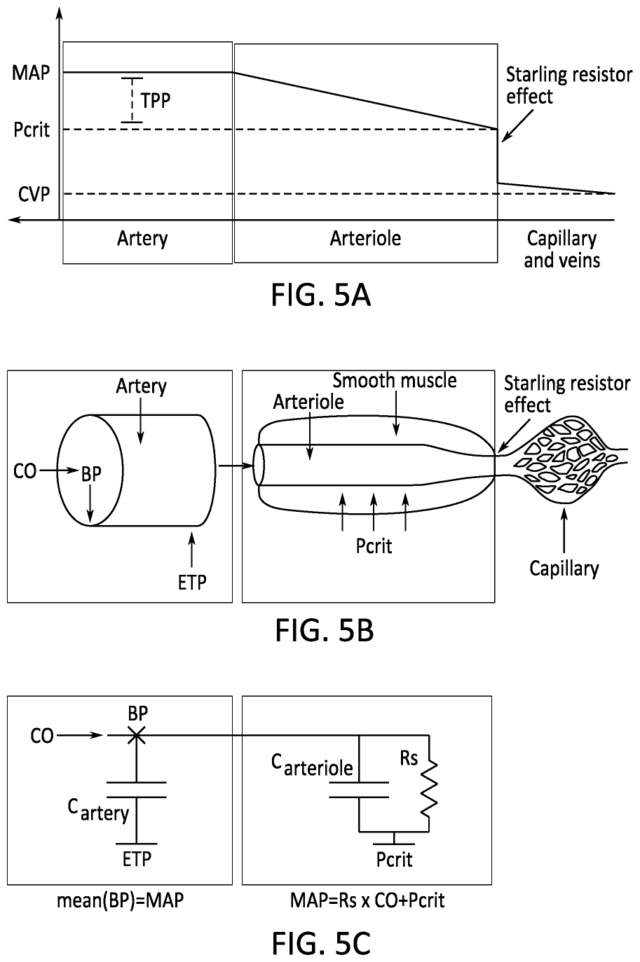

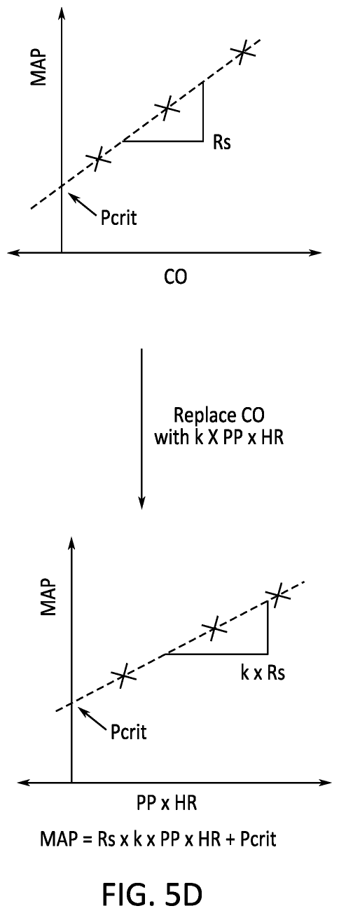

FIGS. 5A-5D demonstrate how Pcrit and TPP describe the state of the systemic arterial circulation. FIG. 5A provides a schematic showing that the vasculature behaves as a Starling resistor, with circulatory collapse at the distal end of the arteriole when MAP falls below Pcrit, resulting in zero flow. TPP=MAP−Pcrit is defined as the pressure difference driving flow through the circulation and Pcrit is typically higher than CVP. FIG. 5B shows a schematic of a physiological model showing Pcrit at the level of the arterioles impacting vascular smooth muscle tone. FIG. 5C demonstrates a lumped parameter circuit representation of the model in FIG. 5B. These models allow for the effect of an external tissue pressure (ETP) on large arteries separately from the impact of Pcrit on the arterioles. MAP can be calculated at steady state using Ohm's law as MAP=Rs×CO+Pcrit, where Rs represents the Starling resistance. C_{artery} and Cₐᵣₜₑᵣᵢₒₗₑ represent arterial and arteriolar compliance, respectively. FIG. 5D demonstrates that Pcrit can be estimated from a plot of CO versus MAP at two or more conditions. A simplified estimate of CO as PP multiplied by HR allows this estimate to be made from arterial blood pressure data alone, without a separate CO measurement. k represents a scale factor between CO and PP×HR.

The systemic circulation can be modeled as a Starling resistor, with Pcrit defined as the arterial pressure below which blood flow stops. Pcrit is generally higher than CVP, resulting in a “waterfall edge” to the arterial pressure whereby changes in CVP do not impact TPP, measured as MAP-Pcrit, as shown in FIG. 5A. To explain the role of Pcrit and the concept of TPP, a physiological model of the systemic vasculature can be defined, with terms defined for large and small vessels, as shown in FIG. 5B, and a lumped parameter circuit model shown in FIG. 5C. At steady state, CO is the flow through the systemic circulation, which generates a MAP in proportion to the magnitude of the Starling resistor (Rs). TPP can then be defined as the pressure drop across Rs, which equals MAP minus Pcrit. Under most scenarios, external tissue pressure (ETP) can be considered a function of the intrathoracic or intra-abdominal pressure and would impact the compliance of large vessels, whereas Pcrit is largely impacted by vascular tone at the level of smaller arterioles. Notably, the hemodynamic measurement SVR, which can be defined as (MAP−CVP)/CO, is not required in this model because the Starling resistor effect redefines the pressure drop across the circulation.

Estimation of Pcrit from Arterial Blood Pressure Waveforms

Pcrit can be estimated in a patient with intact circulation by knowing at least two points on a plot of CO versus MAP (FIG. 5D) and extrapolating the best-fit line to zero CO. An assumption here is that the resistance (Rs) at the time of measurement of CO and MAP remains fixed. Acquisition of these data in standard clinical care would typically require specialized equipment and maneuvers to modulate CO. To simplify, CO can be approximated as pulse pressure (PP) multiplied by heart rate (HR). This approximation was tested by comparing changes in PP*HR with changes in measured CO; concordance was found consistent with that seen between other CO estimation methods. In the limit that pulsatile flow goes to zero, PP*HR also goes to zero, and the Pcrit pressure intercept on a PP*HR versus MAP plot should be the same as when using paired CO and MAP measurements. Thus, Pcrit can be estimated continuously from available arterial blood pressure (ABP) monitoring devices such as an indwelling arterial catheter.

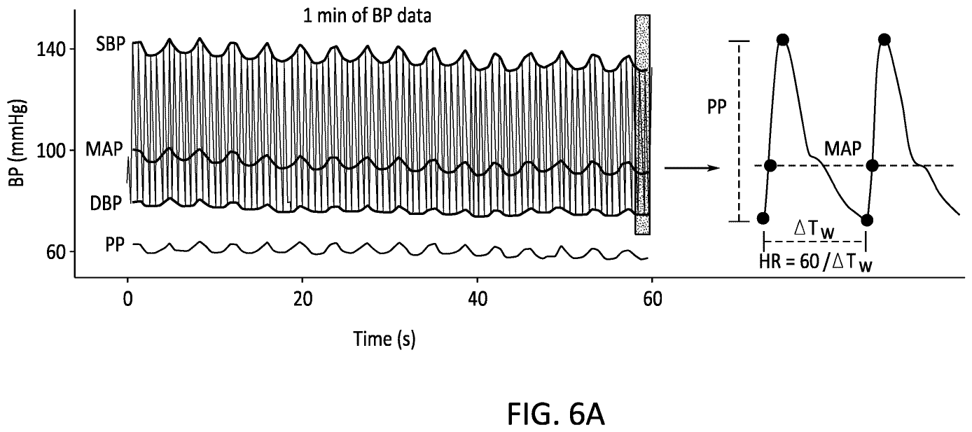

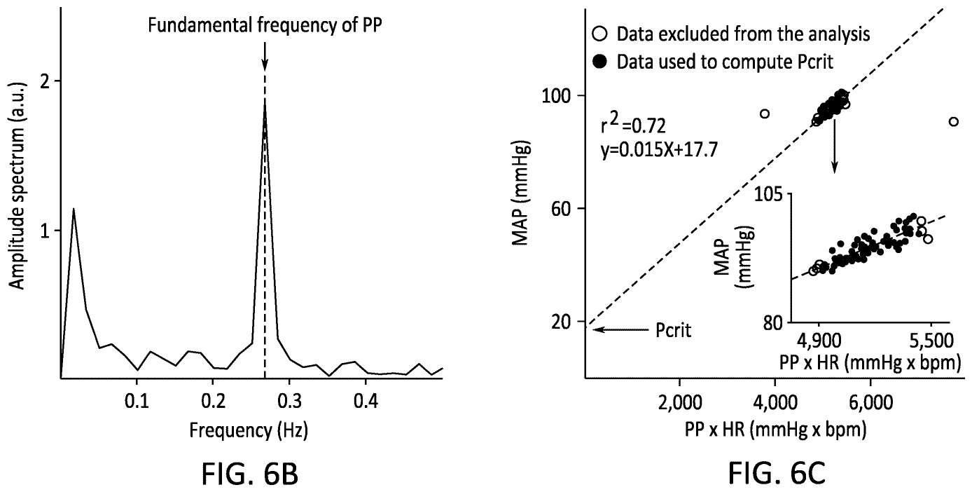

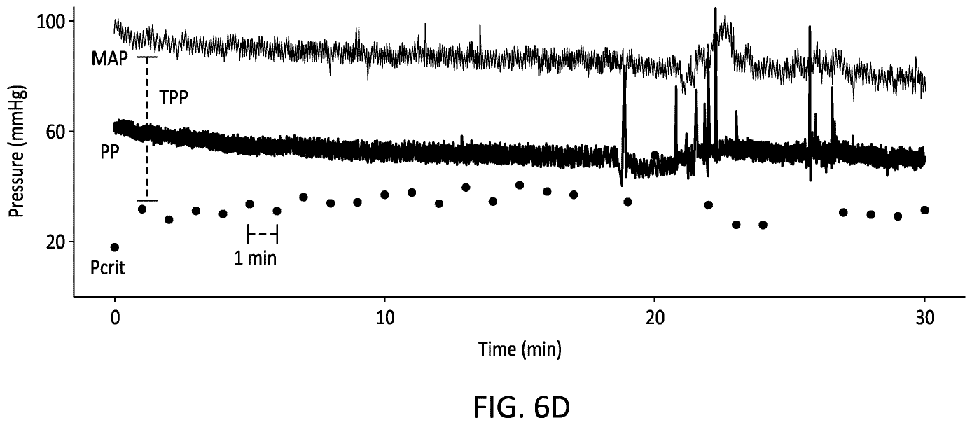

Referring to FIGS. 6A-6D, the disclosed approach allows Pcrit and TPP to be measured continuously with high temporal resolution from ABP data. FIG. 6A provides example blood pressure data from an arterial catheter showing that natural variation in beat-to-beat BP occurs over short timescales, resulting in modulation of systolic blood pressure (SBP), MAP, diastolic blood pressure (DBP) and PP over seconds to minutes. PP can be defined as SBP−DBP and ΔTw can be defined as the cardiac cycle length measured between two consecutive DBP values. FIG. 6B shows a frequency spectrum from a Fourier transformation of the PP data in FIG. 6A, with fundamental frequency equal to the respiration rate. The mean of the signal was subtracted before computing the Fourier transform to facilitate display. FIG. 6C shows a plot of MAP versus PP×HR for the defined 1-min time interval of data in a scatter cloud of data that could be fitted with a line to determine the pressure axis intercept representing Pcrit. The coefficient of correlation (r²) was used to quantify the accuracy of the fit, with r²>0.3 taken as a threshold to determine Pcrit. FIG. 6D shows a plot of continuous MAP and PP data over time along with the serial calculations of Pcrit and TPP, defined as MAP-Pcrit.

Still referring FIGS. 6A-6D, the disclosed approach can be performed using at least two points measured at similar resistance Rs. To achieve this, natural variability in ABP can be leveraged over short periods of time. As shown using data from an ICU patient in FIG. 6A, there is significant variation over seconds to minutes in the pressures. On the shortest scale, the variability results from respiration-induced changes in ventricular filling (preload) or heart rate that leads to stroke volume and CO variation. Over longer time scales, autonomic contributions to HR and vascular tone can drive pressure changes. FIG. 6B demonstrates the frequency spectrum of the PP waveform with respiration as the dominant frequency. Using beat-to-beat detection of PP and cardiac cycle length (1/HR), every beat of the heart can contribute data to the PP*HR versus MAP plot. Over a predefined interval where resistance is assumed constant, these data create a scatterplot of the variability, as shown in FIG. 6C. In an example provided, a time interval of 1 minute was selected for analysis, which provides a compromise between high time resolution and sufficient data points for reasonable fitting. However, other measurement strides can be used. A linear fit to the scatterplot can be determined, and the zero-flow intercept recorded as Pcrit. Using this approach, Pcrit and TPP could be calculated every minute (or other stride length) for each patient, as shown in FIG. 6D. In the examples provided, this analysis was applied to ABP waveforms from the first 24 hours of data for nearly 6000 patients in a cardiac surgical ICU, resulting in almost 144,000 hours of analyzed data. Comparison of Pcrit estimates over different time windows, showed relative stability between 30 sec and 2 min and variation in the linear fit over time intervals of many minutes consistent with changing Rs and Pcrit over longer periods. Over our selected 1-minute interval, high quality linear fit was achieved over a range of MAP and PP*HR variation, and variability in both PP and HR contributed to the estimates of Pcrit. Complete details of example algorithms and analyses are provided in below.

### Methods

### Study Design and Primary Dataset

The study was performed under a protocol approved by the Institutional Review Board at the Massachusetts General Hospital. A retrospective analysis was performed on data collected from a cohort of patients in the cardiac surgical intensive care unit (ICU) at the Massachusetts General Hospital (MGH).

This population was chosen because of the availability of standard invasive hemodynamic measurements for comparison, including pulmonary artery catheter data, and because of the availability of well-adjudicated outcomes data as part of the MGH institutional Society for Thoracic Surgery (STS) database. Patients admitted to the ICU with indwelling radial, brachial, or femoral artery blood pressure (BP) catheters, during the period of Nov. 7, 2015 to Jun. 14, 2021, were included in this investigation. High frequency sampled waveforms (120 Hz) of the blood pressure were available for all included patients. The BP waveforms were part of a research archive consisting of waveform data collected and saved from bedside telemetry monitors. The study analyses also utilized laboratory data, vital signs, and discrete hemodynamic measurements taken from the hospital's electronic health record and electronic data warehouse.

Data during the first 24 hours of admission to the ICU and the adjudicated outcomes data (from the STS database) were used for analyses described herein. A cohort of 6804 admissions to the MGH cardiac surgical ICU (6591 patients) was identified with appropriate waveform data, including data from multiple admissions of the same individuals (N=200). A total of 5729 admissions (5521 patients) had well-adjudicated outcomes from the STS database.

Code for data processing and analysis was written in Python using open-source libraries including scikit-learn, NumPy, and SciPy.

### External Validation Dataset

A second independent dataset was used for external validation of analyses performed on the primary dataset. This dataset was obtained from the publicly available Medical Information Mart for Intensive Care (MIMIC) III database, which contains high-frequency arterial line waveform data (125 Hz) for patients in the cardiac ICU matched to select electronic health record information such as demographics, vital signs, and laboratory reports, as well as available outcomes data for length of stay in the hospital and in-hospital mortality. Data in MIMIC-III was collected between 2001 to 2012. Institutional Review Boards of the Beth Israel Deaconess Medical Center (2001-P-001699/14) and the Massachusetts Institute of Technology (no. 0403000206) approved the use of MIMIC-III data collection protocol.

We identified a total of 878 ICU admissions (860 patients, age=71.3±31.5 years, female=33.6%, mortality=3.75%) to the cardiac surgery ICU with continuous arterial blood pressure waveforms available during the first 24 hours of their stay in the ICU and with available outcomes data for length of stay and mortality. From these ICU admissions, records with poor quality blood pressure waveforms were excluded, leaving a total of 864 ICU admissions (846 patients) for final analysis. We did not have access to formally adjudicated STS outcomes for the MIMIC cohort. Length of stay in the hospital, in-hospital mortality, and the lactate values were available, however, from the matched clinical records of the patient in the MIMIC-III database. Length of stay was calculated as the time spent in the hospital since the admission into the cardiac ICU, and the lactate value was determined as the maximum lactate value during the patients' first 24 hours in the ICU.

### Detection of Beat-to-Beat Features (PP, MAP, HR) of a BP Waveform

The BP waveforms recorded at the radial, brachial, or femoral arteries were used to measure systolic blood pressure (SBP), diastolic blood pressure (DBP), pulse pressure (PP), mean arterial pressure (MAP), and heart rate (HR) as illustrated in FIG. 6A. From the primary dataset, radial or brachial ABP waveforms were available for 6008 ICU admissions, and femoral BP was used for those individuals (N=79) without an upper extremity BP waveform. A combination of femoral and radial BP waveforms was used for analysis on 796 ICU admissions due to the absence of upper extremity BP waveform for short intervals of time. In the external dataset, ABP waveforms from 864 admissions were used.

Maximum and minimum locations, detected from every cardiac cycle of the BP waveform were used to calculate the PP, MAP, and HR beat by beat during the first 24 hours in the ICU (see FIG. 6A). The maximum and minimum locations of the BP waveform were detected as follows. First, the waveforms were low-pass filtered with a cutoff frequency of 2 Hz to remove high-frequency signals. Then, minima and maxima from these filtered waveforms were used as an initial estimate to locate the maximum and minimum of every cardiac beat. Specifically, a window of 100 ms, centered on the initial estimate, was assessed on the unfiltered waveform to locate the true maximum and minimum locations. A single cardiac cycle was defined as the BP waveform between two adjacent minima. The height and time average of the BP waveform within a single cardiac cycle was used as PP and MAP, respectively, and the time-width of the cardiac cycle (ΔTw) was used to obtain HR (=60/ΔTw). MAP, PP, and HR were calculated using the above algorithm on BP data from all ICU admissions. The same detection algorithms were applied to both cohorts.

Estimation of Pcrit and TPP from MAP, PP, and HR

Pcrit was estimated from the pressure-axis intercept of the graph between MAP and the product of PP and HR (PP*HR). The intercept was obtained by fitting a line to the plot of MAP and PP*HR (see FIG. 6C). MAP, PP, and HR obtained from all cardiac cycles within a time window of 1 minute were used to estimate a single value of Pcrit. The Pcrit estimation algorithm was iteratively repeated (stride=1 minute) on all the data recorded for a duration of 24 hours. The scikit learn library from Python was used to implement the linear fit, and NumPy was used for computations.

A set of rules were followed to improve the robustness of Pcrit estimation. First, PP*HR data points outside the 5 to 95 percentiles were removed to filter extreme outliers that may be due to sources of measurement artifact. Second, estimated Pcrit values were discarded if the slope or pressure-axis intercept of the best-fit line were negative, or the coefficient of determination (r²) of the fit was less than 0.3. By default, 0.3 was used as the threshold for r² based filtering unless specified otherwise in a specific analysis. Once Pcrit was calculated for every 1-minute time window, TPP was estimated as the difference between the average MAP over the 1-minute interval and the estimated Pcrit value.

From the analyzed 6804 ICU admissions in the primary dataset, 6693 admissions had a valid TPP value estimated based on the above rules. 111 ICU admissions did not have a single value of Pcrit or TPP estimated from the first 24 hours of admission to the cardiac ICU. These individuals had a short duration of BP waveforms with substantial artifact or noise. Another exclusion criterion was imposed on the minimum number of TPP values detected from an individual; ICU admissions with TPP values estimated for a duration of less than 1 hour were excluded from further analysis. Based on these inclusion/exclusion rules, Pcrit and TPP estimated from a cohort of 5988 ICU admissions (5532 patients) from the primary dataset were used in all analyses described herein.

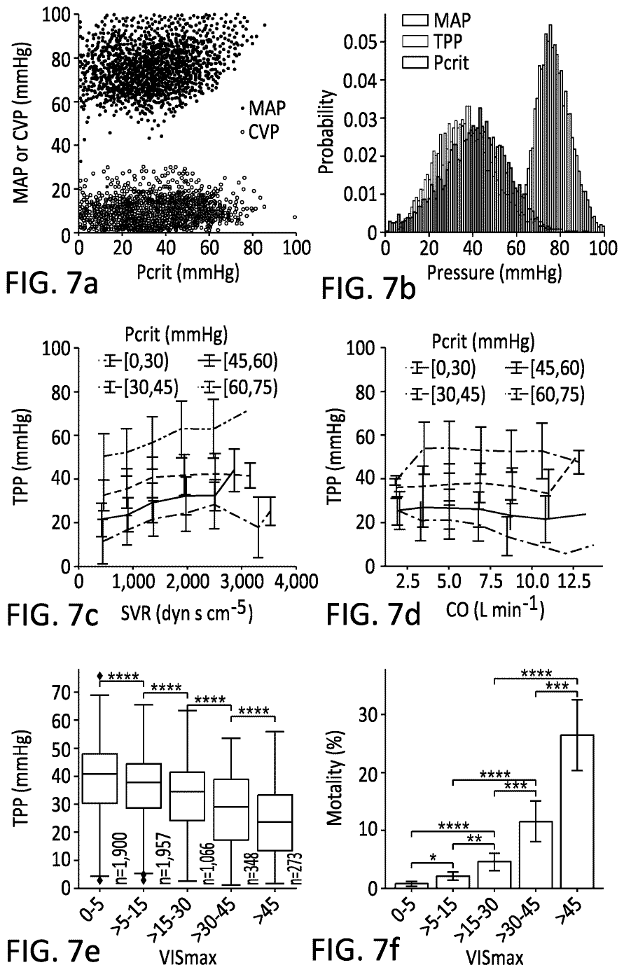

Pcrit values estimated from the above algorithm were compared with the Central Venous Pressure (CVP) and MAP of the patient population over the first 24 hours in the cardiac ICU. In addition, the relationship between TPP, systemic vascular resistance (SVR), and cardiac output (CO) was investigated. CVP and CO were measured using a central catheter and the thermodilution technique, respectively. Values of SVR were obtained from those recorded in the electronic health record according to clinical standard calculations. Patients without a CVP measurement, or those CVP datapoints of a patient with a magnitude of more than 30 mmHg (assumed to be inaccurate) were excluded from the analysis. Also, Pcrit was estimated from the linear fit of MAP and PP*HR with an r²>0.5 taken as a minimum threshold in order to provide higher accuracy of comparisons with other hemodynamic parameters in FIGS. 7A, 7C and 7D. Based on the exclusion criteria, a cohort of 1911 ICU admissions was included in the final analysis presented in FIGS. 7A, 7C, and 7D. FIG. 7A shows all the paired values (5438) of CVP, MAP, and Pcrit for these ICU admissions. SVR, CO, and TPP from these patients were grouped based on Pcrit into four cohorts—Group 1: 0≤Pcrit<30 mmHg; Group 2: 30≤Pcrit<45 mmHg; Group 3: 45≤Pcrit<60 mmHg; Group 4: 60≤Pcrit<75 mmHg, as illustrated in FIGS. 7C and 7D. The mean TPP, SVR and CO as well as +/−one standard deviation of the data for each Pcrit group are shown.

The average Pcrit, MAP and TPP values estimated from the first hour of the data available from the cohort of 5988 ICU admissions were studied to investigate the relationship between the distribution of these variables. Pcrit measurements with r²>0.5 were included for analysis. A set of 5514 ICU admissions were identified based on these criteria, and the resulting histogram of Pcrit, MAP, and TPP is illustrated in FIG. 7B. The above analysis was repeated after stratifying the patient data based on gender.

### Vasoactive-Inotropic Score (VIS) Analysis

The impact of vasoactive drugs on the TPP was evaluated, as illustrated in FIGS. 7E-7F, using the vasoactive-inotropic score (VIS). Out of the 5988 ICU admissions investigated, 5544 were included in the analysis to compare VIS and TPP. Patients without medication records corresponding to the first 24 hours of their postoperative period in the ICU were excluded from the analysis. VIS was calculated according to:

$$VIS = 10000 \times Vasopressin dose ( in U / kg / min ) + 100 \times Epinephrine dos e ( in μg / kg / min ) + 100 \times Norepinephrine dose ( in μg / kg / min ) + 50 \times Levosimendan dose ( in μg / kg / min ) + 25 \times Olprinone dose ( in μg / kg / min ) + 20 \times Methylene blue dose ( in mg / kg / hr ) + 10 \times Melrinone dose ( in μg / kg / min ) + 10 \times Phenylephrine dose ( in μg / kg / min ) + 10 \times Terlipressin dose ( in μg / min ) + 0.25 \times Angiotensin II dose ( in ng / kg / min ) + 1 \times Dobutamine dose ( in μg / kg / min ) + 1 \times Dopamine dose ( in μg / kg / min ) + 1 \times Enoximone dose ( in μg / kg / min ) .$$

This formula accounts for a range of possible medications, with most patients receiving only a small fraction of these and norepinephrine being most common. The maximum value of VIS evaluated for the first 24 hours was compared with the respective mean TPP of the patient during the same period. Five categories of VIS were defined according to Group 1: 0<VIS≤5; Group 2: 5<VIS≤15; Group 3: 15<VIS≤30; Group 4: 30<VIS≤45, and Group 5: 45<VIS. Note that the above study was also performed by comparing the mean value of VIS to the average TPP over the first 24 hours in the ICU. The mean value gives a better estimate of the total exposure that the patient has had to vasoactive medications.

In all comparative studies discussed here, a standard list of additional outcomes, including patient mortality, reoperation rate, prolonged ventilation, and length of stay (LoS), were investigated for every VIS group. Patient mortality, reoperation rate, and prolonged ventilation data were obtained from the MGH STS database (binary adjudicated outcomes), and LoS was calculated as the time spent in the hospital since admission to the cardiac ICU after the surgery. All patients declared dead, regardless of cause, during hospitalization or before the end of the 30th postoperative day after discharge from the hospital were used for calculating patient mortality. Reoperation due to any cardiac reason, tamponade, or bleeding were utilized while computing reoperation rate. An individual with a ventilation or reintubation time of more than 24 hours was included in the list of patients with prolonged ventilation. The percentage patient mortality of a VIS group was calculated as the ratio of individuals dead over the total number of ICU admissions in the group having information in the STS database. A similar approach was used to calculate the percentage of individuals that needed reoperation and prolonged ventilation in each VIS group. The average LoS of individuals in each group was also calculated.

Standard box plots were used to show the distribution of TPP values in each VIS group. A one-side ANOVA test followed up by Tukey's HSD test for multiple-comparisons was performed to compare the TPP distribution for each VIS group. The mean of outcomes for individuals in these groups were represented via bar charts, and 95% confidence interval was used to indicate mean variation. The 95% confidence interval and p-values for the binary outcomes (patient mortality, reoperation rate, and prolonged ventilation data) were calculated using the Bernoulli distribution and chi-square test, respectively. The T-distribution and one-side ANOVA test followed up by Tukey's HSD test for multiple-comparisons were used to calculate the 95% confidence interval and p-value for the mean value of the LoS for these groups, respectively.

### Clinical Outcomes Analyses: Mortality, Length of Stay, Blood Lactate

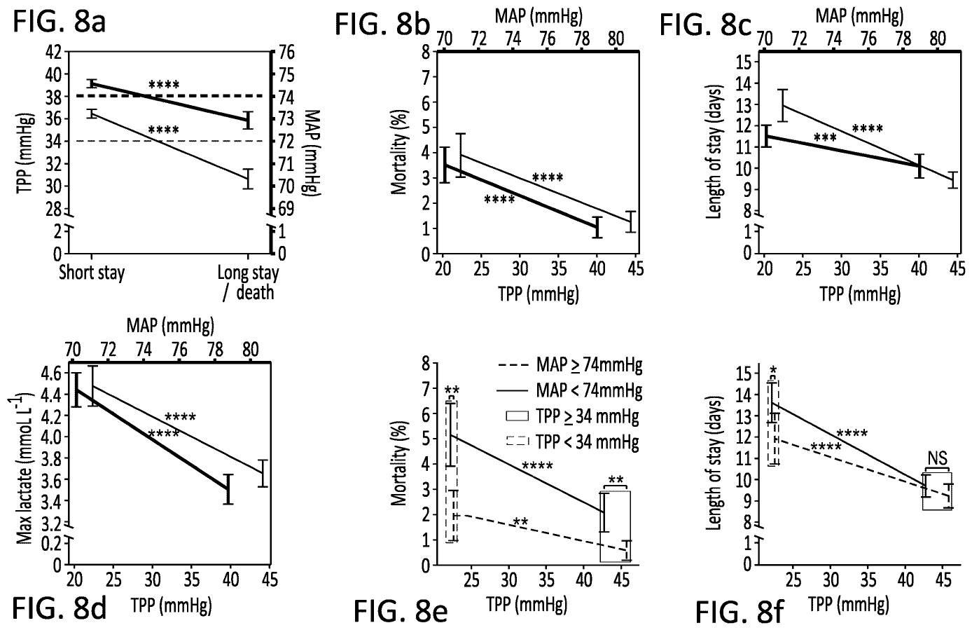

The additive value of TPP as a target for therapy was investigated using a primary cohort of 5988 ICU admissions. The mean value of patients' TPP over the first 24 hours in the cardiac ICU and the mortality, LoS, and maximum blood lactate levels were compared, as illustrated in FIGS. 8A-F. In addition to other variables as discussed earlier, cardiac index (CI) was used for the analysis discussed herein. CI, determined as a ratio of cardiac output over patient's body surface area, is used routinely in clinical care and was available from measurements recorded in the hospital electronic health record. Individuals without CI, MAP, or TPP measurements over the stipulated time were excluded from the analysis, leaving a total of 4899 ICU admissions for the current analysis.

The average CI, TPP, and MAP over the first 24 hours in the cardiac-ICU were used for the following analyses. First, patients were stratified based on their CI into three groups according to: Group 1 (low CI): 1≤CI<2.5 Lmin⁻¹ m⁻², Group 2 (normal-high CI): 2.5≤CI<6 Lmin⁻¹ m⁻², and Group 3 (all patients): 1<CI<2.5 Lmin⁻¹ m⁻². Patients in each of the above groups were further categorized into two groups based on LOS according to: Group A: LoS≤14 days and Group B: a composite of LoS>14 days or death. For Groups 1 to 3, the optimal TPP and MAP values that separate Group A from B were obtained via logistic regression.

Logistic regression on Groups A and B was implemented as follows. First, a balanced dataset was created from the groups. For instance, among the cohort with a low CI (<2.5 Lmin⁻¹ m⁻²), 1092 and 332 subjects were identified in Groups A and B, respectively. Then, 332 random ICU admissions were located from Group A to create a balanced dataset with Group B. Next, a logistic regression model was trained on MAP and TPP values of this balanced dataset to obtain a threshold for optimal separation. The scikit learn library from Python was used to implement the logistic regression model. Multiple optimal TPP and MAP thresholds were obtained via logistic regression by selecting 5 different and random sets of 332 ICU admissions from Group A. Finally, an average of the optimal MAP and TPP, rounded to the closest integer, was determined. The above method to calculate the optimal threshold was implemented separately on the cohorts in Groups 1-3. Average optimal thresholds of 34 and 74 mmHg were determined independently for TPP and MAP, respectively, for the Groups 1-3. A one-side ANOVA test was used to compute the p-values for optimal threshold analyses.

Patients in Group 3 representing all patients were stratified into subgroups above and below the optimal MAP and TPP thresholds and outcomes were compared as shown in FIGS. 8B-8D. Patients in Groups 1-3 were further stratified into additional subgroups based on both optimal MAP and TPP according to: Group P: TPP<34 mmHg, MAP<74 mmHg; Group Q: TPP<34 mmHg, MAP≥74 mmHg; Group R: TPP≥34 mmHg, MAP<74 mmHg; and Group S: TPP≥34 mmHg, MAP≥74 mmHg. The average and 95% confidence intervals for outcomes of mortality, LoS, and maximum lactate for the abovementioned subgroups were calculated and statistical comparisons made (see FIGS. 8B-8F). A chi-square test for independence was used to compute the p-values comparing mortality of different groups and a one-side ANOVA test was used to compute the p-value for the lactate and length of stay analyses. The confidence intervals for continuous variables were estimated using the T-distribution and for binary variables using the Bernoulli distribution.

The outcomes analyses were repeated for the MIMIC external validation cohort using identical methods. Stratification by the MGH optimal thresholds was used for outcomes comparison for true external validation. Outcomes of mortality, LoS, and maximum lactate levels were used. Additional stratification by CI for the external cohort was not possible due to the lack of sufficient available CI data for the cohort.

### Clustering Analyses

The relationship between TPP, MAP, arterial blood lactate concentration (ABLC), and CO was investigated on 5988 ICU admissions recovering from cardiac surgery over the first 24 hours of their post-operative period. Individuals without any of the above measurements over the period of interest were not included. Also, markedly out-of-range measurements for an individual were excluded from the analysis. Specifically, MAP values of more than 120 mmHg or less than 30 mmHg were excluded. ABLC values were restricted to a range of 0 to 20 mmolL⁻¹. CO values of less than 1 Lmin⁻¹ and more than 15 Lmin⁻¹ were ignored. In addition, a TPP measurement of more than 100 mmHg was ignored for the current clustering-based analysis. Based on the above inclusion-exclusion criteria, clustering was performed on a total of 3592 ICU admissions in the current analysis.

For clustering analysis, 24-hour trajectories of all the variables of interest, including TPP, MAP, ABLC, Pcrit, SVR and CO, were created for every patient by averaging values within a time window of 4 hours (stride=4 hours) for a duration of 1 day. Thereby, a 24-hour trajectory of all the variables, defined via a maximum number of 6 datapoints, was obtained for each patient. Special care was needed for variables with missing datapoints in the 24-hour trajectory. Missing datapoints were left undefined and replaced with “Nan” or “not a number” for temporal alignment of the 24-hour trajectories.

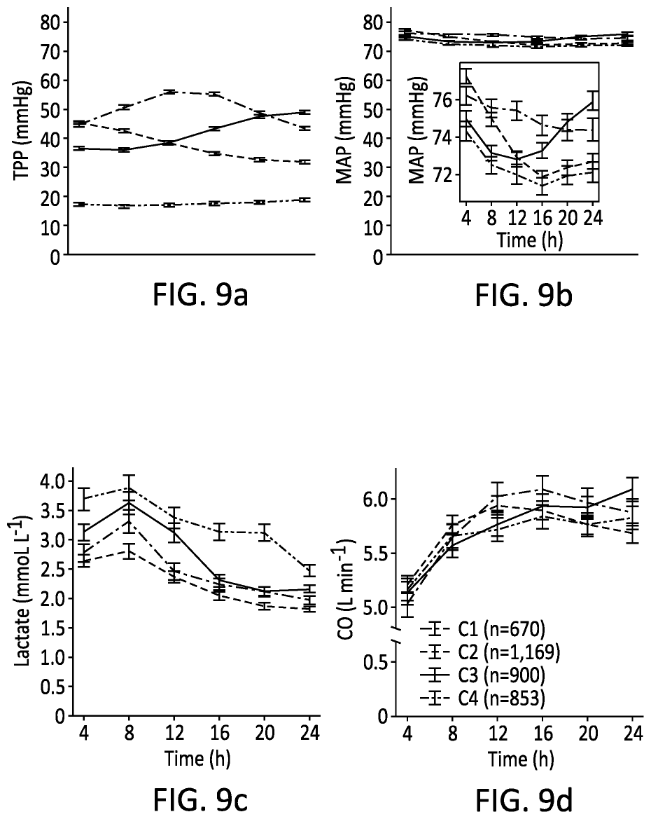

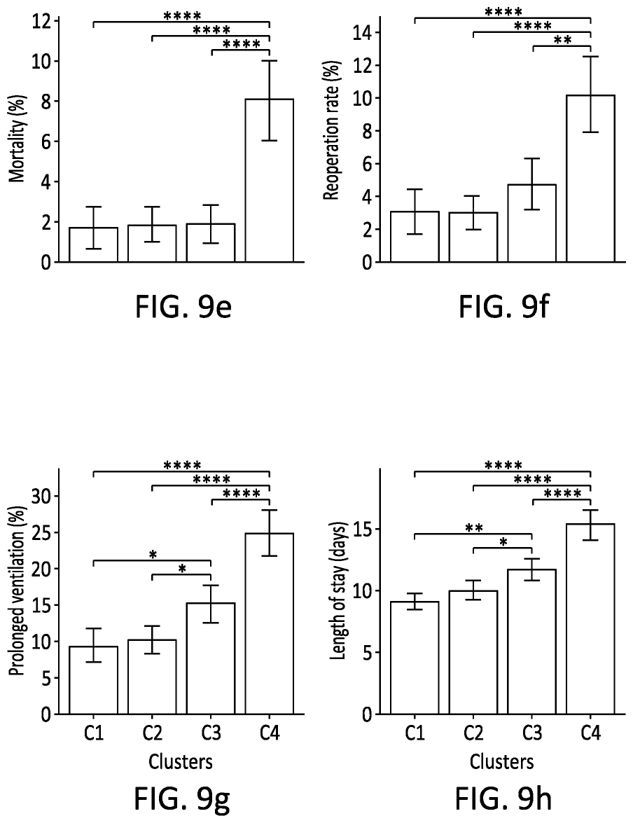

For FIGS. 9A-9H, clustering was performed using the absolute value and the shape of the TPP's 24-hour trajectory. TPP trajectories were organized in the form of a 3592×6 matrix, T, with the trajectory of each patient as a row vector. Then, the T matrix was standardized with the global mean (Gᵤ) and standard deviation (Gₛ) to create a V matrix; V=(T−Gᵤ)/Gₛ. The relative value of the trajectories is encoded in the V matrix. Similarly, a matrix representing the shape, hereby referred to as the S matrix, of the trajectories was created by standardizing every row vector in T with the row's corresponding mean and standard deviation. Finally, a new matrix, SV, was created by concatenating the rows of the S and V matrixes; SV=[S V] such that the value and shape of the trajectory were encoded in a single matrix. A K-means algorithm was applied to the SV matrix to cluster groups of trajectories with unique shapes and values. Only TPP trajectories without any missing data were input into the K-means algorithm. A total of 3592 ICU admissions were available with fully defined TPP trajectories, and the final clustering was performed on these trajectories.

Four main clusters, including C1, C2, C3, and C4, were defined based on the above algorithm. The mean trajectory and 95% confidence interval of the mean are indicated for each of the 4 TPP clusters in FIG. 9A. The T-distribution was used to obtain the 95% confidence interval of the mean. The corresponding MAP, ABLC, and CO trajectories for these clusters were identified and plotted as shown in FIGS. 9B, 9C, and 9D, respectively. STS outcomes were also obtained for all the ICU admissions within each cluster. Three main STS outcomes, including mortality, reoperation rate and prolonged ventilation, were studied for patients in these clusters. In addition, LoS was also included in the outcome analysis. The mean and 95% confidence interval of the mean were plotted for each of the clusters as shown in FIGS. 9E-9H. The confidence interval was calculated using the Bernoulli distribution and T-distribution for all the STS outcomes and LoS computations, respectively. The p-values were determined via a chi-square test with Bonferroni correction for multiple-comparisons and one-side ANOVA followed up by Tukey's HSD test for multiple-comparisons for the STS outcomes and LoS computations, respectively.

### Statistical Methods

Standard statistical methods were used for all the analyses discussed herein. Box plots were utilized to illustrate the distribution of a parameter in a group. The first and third quartiles of the parameter were represented with an upper and lower line of the box plot. The median value of the parameter is shown as the middle line of the box plot. After removing the outliers, the lowest and highest parameter within the group is indicated with solid horizontal lines. Outliers are represented as individual points.

Bar charts were utilized to show a parameter's mean value in a specific group or cluster. The mean value of the variable is indicated via the height of the bar. The error bars on these plots reveal the 95% confidence interval of the mean value. Undefined terms were excluded from the computation of the mean value.

The significance of a parameter compared to the same estimated from different groups was determined using p-values. A p-value less than 0.05 is indicated with a single (*), and the degree of significance increases with the number of (*). For instance, a p-value less than 0.005 is marked with two (*). A maximum of four (*) are displayed for all p-values less than 0.00005.

### Results

### Relationship of TPP and Pcrit to Conventional Hemodynamic Measures

FIGS. 7A-7F demonstrate that Pcrit and TPP can provide unique information compared to conventional hemodynamic metrics. FIG. 7A shows scatterplots of MAP and CVP versus Pcrit (n=1,911 ICU admissions; 5,438 data points). FIG. 7B shows distributions for MAP, Pcrit and TPP for n=5,514 ICU admissions. FIG. 7C provides plots of TPP versus SVR for defined incremental ranges of Pcrit. FIG. 7D plots TPP versus CO for the defined Pcrit ranges. Cohorts for Pcrit groupings were taken as subsets of a larger dataset in a where SVR and CO measurements were available. The groupings were defined as 0≤Pcrit<30, n=2,094 data points; 30≤Pcrit<45, n=2,081 data points; 45≤Pcrit<60, n=1,066 data points; 60≤Pcrit<75, n=183 data points. FIG. 7E plots mean TPP versus VISmax over 24 h after ICU admission for n=5,544 ICU admissions. FIG. 7F shows mortality versus VISmax for the cohort described in FIG. 7E. Pcrit data for FIGS. 7A-7D were selected based on a quality of fit with r²>0.5 to improve accuracy of individual comparisons, while r²>0.3 was used in FIG. 7E to include as many patients as possible. Data points in FIGS. 7C and 7D are shown as the mean±1 s.d. The box plots in FIG. 7E display the first, second and third quartiles with whiskers showing the extent of the distribution (median±1.5 times the interquartile range) and the outliers are represented as individual points. The bar charts in FIG. 7F show the mean±95% CIs of the mean. A one-sided analysis of variance (ANOVA) test followed by Tukey's honestly significant difference (HSD) test for multiple comparisons was performed to compare the TPP distribution for each VIS group in FIG. 7E. The P values for FIG. 7F were calculated using a two-sided chi-squared test with Bonferroni correction for multiple comparisons. Significance levels are indicated as: ****P<0.00005, ***P<0.0005, **P<0.005, and *P<0.05.

Standard management of patients with circulatory shock relies upon invasive hemodynamic measurements including the MAP, CVP, CO, and SVR. FIGS. 7A-7F present data from 1911 patients in the cardiac surgical ICU with time-aligned sets of measurements (5438 sets). As shown in FIG. 7A, Pcrit provides unique information compared to MAP and CVP, with no obvious dependencies between the variables. Whereas MAP in this ICU population follows a tight and approximately normal distribution, Pcrit and TPP have broader and more complex distributions, reflecting underlying heterogeneity in perfusion pressure characteristics of the patients (FIG. 7B). Distributions in Pcrit and TPP were similar for male and female patients, and there was considerable heterogeneity in Pcrit and TPP metrics at all MAP levels. The relationship between TPP and SVR shows a modest linear relationship at low SVR for incremental ranges of Pcrit and flattens out at higher SVR (FIG. 7C). Notably, TPP has a complex relationship with CO, without consistent increase or decrease across a range of CO values and Pcrit intervals (FIG. 7D). With both SVR and CO relationships, there is a stepwise increase in TPP with incrementally lower Pcrit levels. To gain insight into factors that control TPP, the vasoactive inotrope score (VIS) was calculated for patients with available data (N=5544), and VIS was compared it to TPP. The VIS represents a measure of the total vasoactive support required to maintain hemodynamic goals, with higher VIS generally representing more severe circulatory shock. FIG. 7E demonstrates a highly significant relationship between the maximum VIS (VISmax) over 24 hours after ICU admission and the mean TPP, with higher VISmax corresponding to lower mean TPP. Furthermore, VISmax predicts mortality with high significance, reflecting the severity of circulatory shock (FIG. 7F). Risk for additional outcomes, including reoperation, prolonged ventilation, and length of hospital stay, are also stratified with high statistical significance by both maximum and mean VIS over 24 hours. Analyses of subsets of patients with cardiac pacing, acute heart failure, right heart failure, different cardiac surgery procedures, and mechanical ventilation demonstrate the ability for Pcrit and TPP to identify differences in the hemodynamic profiles. Pcrit values estimated from simultaneous upper and lower extremity catheters also highlight similar overall distributions.

### TPP Adds Value to MAP for Risk Prediction

FIGS. 8A-8F demonstrate how TPP can predict outcomes in patients in the cardiac surgical ICU. FIG. 8A provides analysis of the mean TPP and MAP values for patients during the first 24 h in the ICU according to outcomes, with a favorable outcome of short hospital stay (n=4,045 admissions) compared to an unfavorable outcome of long hospital stay or death (n=854 admissions). Logistic regression identified an optimal TPP threshold of 34 mmHg and an optimal MAP threshold of 74 mmHg to separate outcome groups. The dashed lines represent the regression thresholds. FIGS. 8B-8D show comparisons of mortality (FIG. 8B), length of stay (FIG. 8C) and maximum blood lactate value (FIG. 8D) for patients grouped above and below the optimal TPP and MAP thresholds (n=2,919 above the TPP threshold, n=1,980 patients below the TPP threshold; n=2,335 above the MAP threshold, n=2,564 below the MAP threshold). FIGS. 8E-8F show comparisons of the outcomes of mortality (FIG. 8E) and length of stay (FIG. 8F) for groups stratified by both TPP and MAP thresholds (low MAP and low TPP, n=1,209; low MAP and high TPP, n=1,355; high MAP and low TPP, n=771; high MAP and high TPP, n=1,564). Data are displayed as the mean and 95% CIs. P values in FIGS. 8A, 8C, 8D, and 8F were calculated using a one-sided ANOVA. P values in FIGS. 8B and 8E were calculated using a two-sided chi-squared test. P-values are indicated as: ****P<0.00005, ***P<0.0005, **P<0.005, *P<0.05, and NS, not significant.

For patients with hypotension in the ICU, the standard MAP target would be 65 mmHg by most consensus guidelines, although limitations of this goal in various patient populations have been widely discussed. In order to gain intuition on how TPP can impact clinical practice, optimal TPP and MAP thresholds were analyzed that stratify outcomes in the patient cohort. FIG. 8A presents analyses from 4899 patients separated by adjudicated outcomes according to the institutional Society for Thoracic Surgeons (STS) database (see Methods). Patients with short stay (<14 days) were compared against those with a composite of long stay (≥14 days) or death during hospitalization in terms of both average MAP and TPP values over the first 24 hours of post-operative ICU stay. Both MAP and TPP were significantly different between the short stay and long stay/death groupings (p<0.0005). Using logistic regression, an optimal TPP threshold of 34 mmHg and an optimal MAP threshold of 74 mmHg best separated these outcome groups. When stratified by these thresholds for MAP and TPP, differences in mortality, mean length of stay, and maximum lactate for patient groups were highly significant (FIGS. 8B-8D). For both high and low MAP groupings, the mortality and mean length of stay remained highly significantly different between cohorts above and below the TPP threshold (FIGS. 8E-8F). High versus low MAP groupings provided separation with high significance within TPP categories for mortality but not for length of stay. Further stratifying the groups by high and low cardiac index (CO divided by body surface area), TPP continued to show additive value over MAP for separating mortality and length of stay, particularly for the low cardiac index grouping. These analyses highlight that TPP provides additional discriminatory information beyond MAP in critically-ill patients managed with standard of care hemodynamic targets.

External validation was performed using data from the Medical Information Mart for Intensive Care (MIMIC) III database. 864 admissions were identified with ABP waveforms for 24 hours after cardiac surgery and associated outcomes for mortality and length of stay. Algorithms developed on MGH data were applied to MIMIC data to determine MAP, Pcrit, and TPP. Distributions for the MIMIC population showed overall similarities to the MGH population. Optimal thresholds to separate outcomes of short stay versus long stay or death were 73 mmHg for MAP and 36 mmHg for TPP, which are also similar to those derived on MGH data. For true external validation, we used thresholds from the MGH cohort for testing the MIMIC cohort. In the MIMIC cohort, the MAP threshold does not reach statistical significance (taken as p<0.05) in separating groups according to mortality, length of stay, or maximum lactate. TPP however can separate all outcomes with statistical significance. Stratifying further by high and low MAP, TPP still provides statistically significant separation for length of stay in the high MAP group. TPP also has strong trends toward significance in separating mortality for both MAP groups and length of stay for the low MAP group (p≤0.07). Lower significance of separation in the external cohort is likely due to the small size of the cohort (864 vs 4899 in MGH) and to relatively lower length of stay in the MIMIC cohort. External validation analyses reenforce that TPP adds value to MAP.

### Evolution of TPP Trajectories and Relationship to Outcomes

Referring to FIGS. 9A-9H, example data shows that TPP trajectories in response to standard of care therapeutics can identify patient groups with worse outcomes. FIG. 9A shows k-means clustering performed on TPP value and shape trajectories, which identified four distinct mean trajectories in TPP over the first 24 h after cardiac surgery. FIGS. 9B-9D show associated trajectories in MAP (FIG. 9B), blood lactate (FIG. 9C) and CO (FIG. 9D) for each TPP cluster. FIGS. 9E-9H show patient outcomes for mortality (FIG. 9E), reoperation rate (FIG. 9F), prolonged mechanical ventilation (FIG. 9G) and length of hospital stay (FIG. 9H) compared according to TPP cluster. Trajectories display the mean and 95% CIs of the mean at each 4-h increment. The bar charts display the mean and 95% CIs. The P values were determined via a two-sided chi-squared test with Bonferroni correction for multiple comparisons (FIGS. 9E-9G) and one-sided ANOVA followed up by Tukey's HSD test for multiple comparisons (FIG. 9H). The number of ICU admissions in each cluster is illustrated in the inset legend in FIG. 9D.

We evaluated how TPP varies with therapy guided by standard hemodynamic targets. The population consists of patients after cardiac surgery, most of whom underwent cardiopulmonary bypass. Treatment for these patients in the first 24 hours in the ICU consists of optimizing cardiovascular function, including MAP and CO, using a combination of volume resuscitation and vasoactive medications, and optimizing respiratory status. Hemodynamic measurements can be labile as patients frequently have a combination of bleeding, vasodilation, and impaired cardiac function. We organized time series data for each patient with sampling every 4 hours over the first 24 hours of ICU stay, including data for MAP, TPP, CO, and blood lactate. We then performed K-means clustering on TPP trajectories of all patients with full available data (N=3592) to identify four most common paths in the recovery period. To equally weight values and shape, we included both absolute values of TPP and normalized trajectories (see Methods). FIGS. 9A-H present results of the clustering analysis, with each curve representing the mean trajectory for a cluster and the error bars showing 95% confidence intervals. As shown in FIG. 9A, there can be markedly different paths, with some patients having uniformly low TPP, while others have consistently high TPP, and still others have either increasing or decreasing trajectories over the course of recovery. Notably, corresponding MAP trajectories for these clusters have small relative ranges in values (FIG. 9B) but show correlation with the TPP clusters in that the highest TPP cluster has highest MAP and the lowest TPP cluster has lowest MAP (inset). Lower TPP overall correlates with higher lactate values (FIG. 9C), consistent with worse tissue perfusion. Cardiac output trajectories display an expected increase in CO in initial hours as patients are resuscitated (FIG. 9D). However, CO clusters do not separate well based upon TPP, consistent with the analysis of FIGS. 7A-7F. We then looked at adjudicated outcomes for the various clusters using the STS database. Mortality and reoperation rates are significantly different in the lowest TPP cluster (FIGS. 9E-9F). Similarly, clustering by TPP identifies groups with higher incidence of prolonged mechanical ventilation and increased length of stay (FIGS. 9G-9H). For comparison, we performed similar clustering analyses according to MAP value and shape, TPP value alone, Pcrit value and shape, and SVR value and shape. MAP clusters can also separate adverse outcomes but with less power than TPP clusters. Clustering on TPP value alone provided the most potent discrimination of outcomes.

### Continuous TPP Monitoring in Clinical Practice

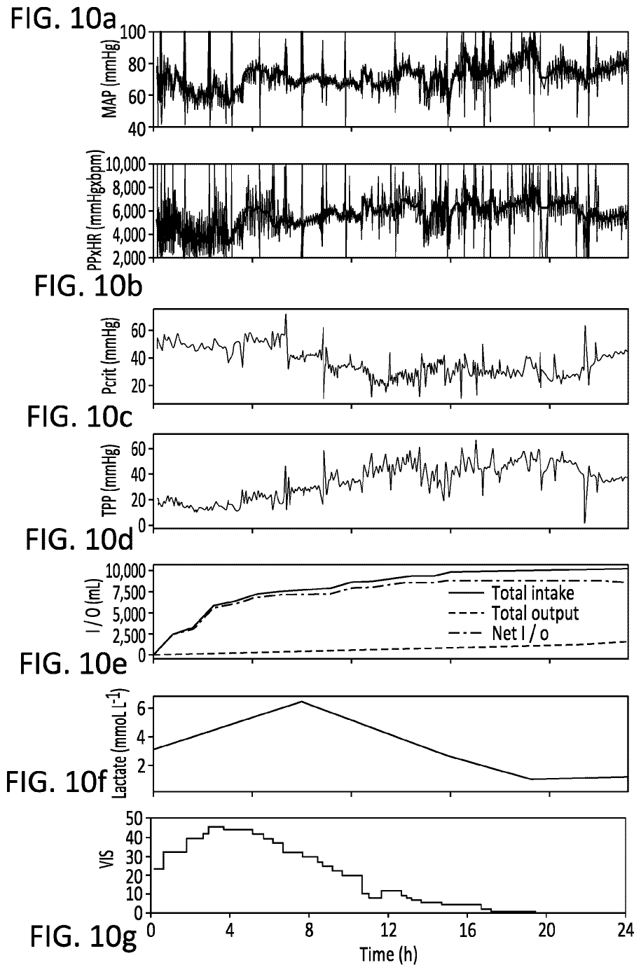

FIGS. 10A-10G, demonstrate how continuous monitoring of Pcrit and TPP provides dynamic hemodynamic information on patients. Individual patient data are shown for the first 24 h of ICU admission from a case of a woman in her seventies who underwent aortic and mitral valve replacements and coronary artery bypass grafting. Data shown include MAP (FIG. 10A), pulse pressure multiplied by HR (PP×HR) (FIG. 10B), Pcrit (FIG. 10C), TPP (FIG. 10D), fluid input/output (I/O) (FIG. 10E), blood lactate levels (FIG. 10F), and VIS (FIG. 10G). The time-averaged trends for MAP and PP×HR are shown with an averaging window size of 20 s.

The method described here offers real-time monitoring of Pcrit and TPP along with MAP and other clinical variables. FIGS. 10A-10G demonstrate an individual patient's hemodynamic time course over 24 hours in the ICU, with Pcrit and TPP calculated with 1-min resolution. This was a woman in her seventies with severe aortic stenosis, calcific mitral valve disease, and coronary artery disease who underwent an aortic valve replacement, mitral valve replacement, and coronary artery bypass grafting. Her post-operative course was complicated by right ventricular dysfunction, atrial fibrillation, and profound circulatory shock and acidosis. She was hemodynamically unstable, as evidenced by the high VIS (FIG. 10G), resulting in high Pcrit (FIG. 10C), and low TPP (FIG. 10D). With volume resuscitation (FIG. 10E), vasoactive support, and resolution of atrial fibrillation she had progressive improvement in hemodynamics after 6 hours, with concomitant decrease in Pcrit and increase in TPP, and a delayed peak and decline in lactate levels (FIG. 10F). Notably, variability in MAP does not fully track that of Pcrit and TPP, reflecting that these metrics provide additional information to distinguish the trajectory and response to therapy. This patient had prolonged length of stay but eventually recovered and was discharged from the hospital. Additional patient vignettes further highlighted the utility of TPP as an additive hemodynamic measure.

### Example Neural Network

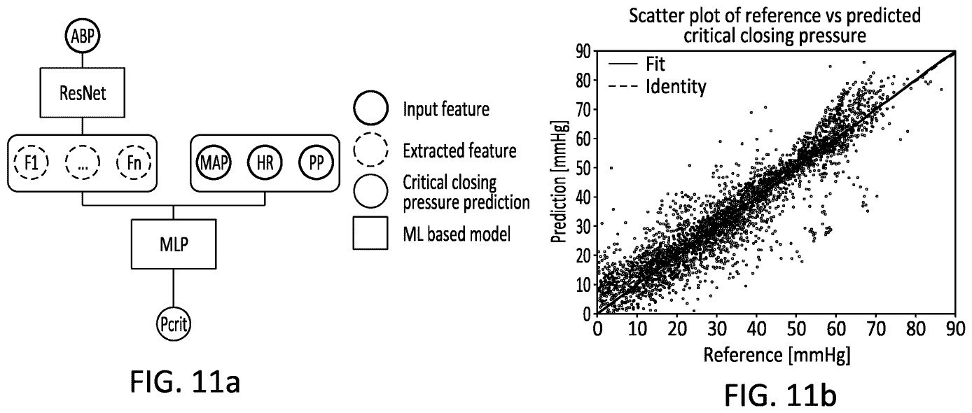

As a non-limiting example, a convolutional neural network was trained to compute Pcrit and TPP based on raw arterial blood pressure (ABP) waveform data. This has the advantage of providing efficient computation for real-time analytics and also allowing ready integration of multiple data types into prediction algorithms for response to therapeutics. FIG. 11A presents a model using a ResNet architecture trained to ingest 60-second segments of ABP waveforms and a multi-layer perceptron (MLP) to integrate the outputs of the ResNet and additional static input features. In this case, the model was trained using 5-fold cross-validation and a training/test/validation split of 0.5/0.25/0.25 from a dataset of 1000 patients and over 1 million Pcrit estimates from cardiac surgical patients using the methods previously described. The model accurately estimates Pcrit with r² of 0.91, with correlation plot shown in FIG. 11B.

Models like this can be optimized for several principal tasks. For example, a model can be trained to provide predictors for response to interventions. In these cases, multi-task training can be used with inputs to include the ABP waveform as well as other variables, such as central venous pressure (CVP) and lactate levels. The outputs can include pre-intervention Pcrit and TPP in addition to the predicted response to intervention (% change or actual values).

This architecture has the benefit of reducing interval steps from raw data to prediction, which will prove useful for real-time analytics.

### Example Noninvasive TPP Prediction

Transfer learning can be used to predict of TPP using a PPG waveform in addition to a standard blood pressure cuff measurement. PPG waveforms incorporate similar beat to beat waveform features and short-time variability that are used to determine TPP and Pcrit without requiring absolute calibration to blood pressure. As shown in FIG. 11B, static BP features can be included in the model to provide calibration to the PPG waveform. As one example, the training data includes thousands of patients with both continuous ABP waveforms and PPG waveforms. Several million paired waveforms segments can be assembled into training and test sets for transfer learning. The CNN can first be trained as above on ABP alone to predict Pcrit. The network can also be fine-tuned and retrained on PPG waveforms and isolated blood pressure measurements alone.

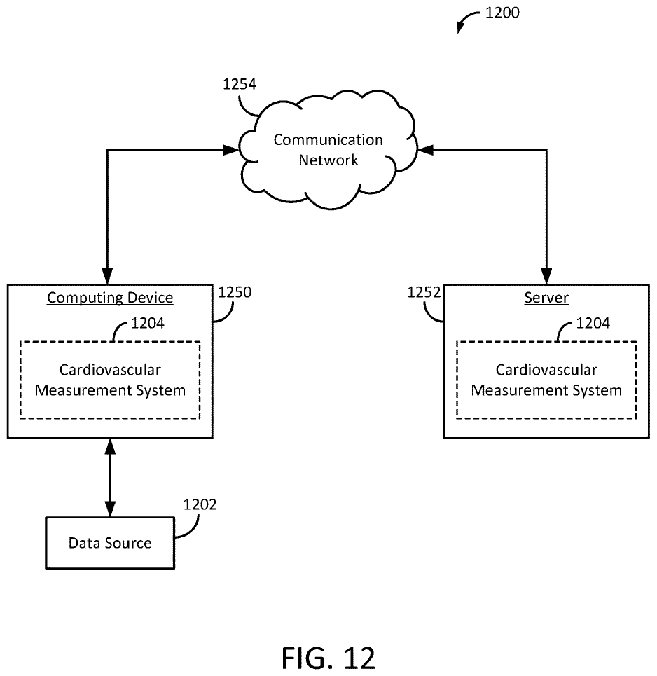

Referring now to FIG. 12, an example of a system 1200 is shown, which may be used in accordance with some aspects of the systems and methods described in the present disclosure. As shown in FIG. 12, a computing device 1250 can receive one or more types of data (e.g., physiological patient data, blood pressure data, treatment data, patient data, and so forth) from data source 1202. In some configurations, computing device 1250 can execute at least a portion of a cardiovascular measurement system 1204 to calculate Pcrit or TPP using the methods described herein. In some configurations, the cardiovascular measurement system 1204 can implement an automated pipeline to characterize cardiac function, process physiological patient data, provide treatment recommendations, generate reports, provide automated treatment, and so forth.

Additionally or alternatively, in some configurations, the computing device 1250 can communicate information about data received from the data source 1202 to a server 1252 over a communication network 1254, which can execute at least a portion of the cardiovascular measurement system 1204. In such configurations, the server 1252 can return information to the computing device 1250 (and/or any other suitable computing device) indicative of an output of the cardiovascular measurement system 1204.

In some configurations, computing device 1250 and/or server 1252 can be any suitable computing device or combination of devices, such as a desktop computer, a laptop computer, a smartphone, a tablet computer, a wearable computer, a server computer, a virtual machine being executed by a physical computing device, and so on. The computing device 1250 and/or server 1252 can also filter data or reconstruct images from the data.

In some configurations, data source 1202 can be any suitable source of data (e.g., measurement data, stored data, user input data, processed data, filtered data), such as a patient monitor, a computing device (e.g., a server storing measurement data or processed data), and so on. In some configurations, data source 1202 can be local to computing device 1250. For example, data source 1202 can be incorporated with computing device 1250 (e.g., computing device 1250 can be configured as part of a device for measuring, recording, estimating, acquiring, or otherwise collecting or storing data). As another example, data source 1202 can be connected to computing device 1250 by a cable, a direct wireless link, and so on. Additionally or alternatively, in some configurations, data source 1202 can be located locally and/or remotely from computing device 1250, and can communicate data to computing device 1250 (and/or server 1252) via a communication network (e.g., communication network 1254).

In some configurations, communication network 1254 can be any suitable communication network or combination of communication networks. For example, communication network 1254 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some configurations, communication network 1254 can be a local area network, a wide area network, a public network (e.g., the Internet), a private or semi-private network (e.g., a corporate or university intranet), any other suitable type of network, or any suitable combination of networks. Communications links shown in FIG. 12 can each be any suitable communications link or combination of communications links, such as wired links, fiber optic links, Wi-Fi links, Bluetooth links, cellular links, and so on.

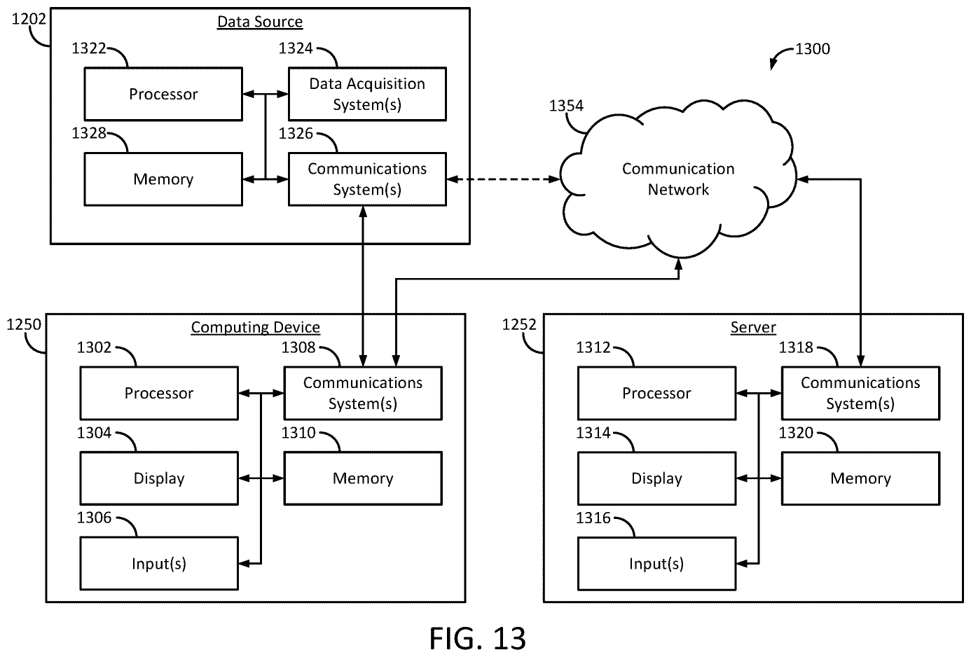

Referring now to FIG. 13, an example of hardware 1300 that can be used to implement data source 1202, computing device 1250, and server 1252 in accordance with some configurations of the systems and methods described in the present disclosure is shown.

As shown in FIG. 13, in some configurations, computing device 1250 can include a processor 1302, a display 1304, one or more inputs 1306, one or more communication systems 1308, and/or memory 1310. In some configurations, processor 1302 can be any suitable hardware processor or combination of processors, such as a central processing unit (“CPU”), a graphics processing unit (“GPU”), and so on. In some configurations, display 1304 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some configurations, inputs 1306 can include any suitable input devices and/or sensors that can be used to receive user input, such as a keyboard, a mouse, a touchscreen, a microphone, and so on.

In some configurations, communications systems 1308 can include any suitable hardware, firmware, and/or software for communicating information over communication network 1254 and/or any other suitable communication networks. For example, communications systems 1308 can include one or more transceivers, one or more communication chips and/or chip sets, and so on. In a more particular example, communications systems 1308 can include hardware, firmware, and/or software that can be used to establish a Wi-Fi connection, a Bluetooth connection, a cellular connection, an Ethernet connection, and so on.

In some configurations, memory 1310 can include any suitable storage device or devices that can be used to store instructions, values, data, or the like, that can be used, for example, by processor 1302 to present content using display 1304, to communicate with server 1252 via communications system(s) 1308, and so on. Memory 1310 can include any suitable volatile memory, non-volatile memory, storage, or any suitable combination thereof. For example, memory 1310 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some configurations, memory 1310 can have encoded thereon, or otherwise stored therein, a computer program for controlling operation of computing device 1250. In such configurations, processor 1302 can execute at least a portion of the computer program to present content (e.g., images, user interfaces, graphics, tables), receive content from server 1252, transmit information to server 1252, and so on. For example, the processor 1302 and the memory 1310 can be configured to perform the methods described herein.

In some configurations, server 1252 can include a processor 1312, a display 1314, one or more inputs 1316, one or more communications systems 1318, and/or memory 1320. In some configurations, processor 1312 can be any suitable hardware processor or combination of processors, such as a CPU, a GPU, and so on. In some configurations, display 1314 can include any suitable display devices, such as an LCD screen, LED display, OLED display, electrophoretic display, a computer monitor, a touchscreen, a television, and so on. In some configurations, inputs 1316 can include any suitable input devices and/or sensors that can be used to receive user input, such as a keyboard, a mouse, a touchscreen, a microphone, and so on.

In some configurations, communications systems 1318 can include any suitable hardware, firmware, and/or software for communicating information over communication network 1254 and/or any other suitable communication networks. For example, communications systems 1318 can include one or more transceivers, one or more communication chips and/or chip sets, and so on. In a more particular example, communications systems 1318 can include hardware, firmware, and/or software that can be used to establish a Wi-Fi connection, a Bluetooth connection, a cellular connection, an Ethernet connection, and so on.

In some configurations, memory 1320 can include any suitable storage device or devices that can be used to store instructions, values, data, or the like, that can be used, for example, by processor 1312 to present content using display 1314, to communicate with one or more computing devices 1250, and so on. Memory 1320 can include any suitable volatile memory, non-volatile memory, storage, or any suitable combination thereof. For example, memory 1320 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 1320 can have encoded thereon a server program for controlling operation of server 1252. In such configurations, processor 1312 can execute at least a portion of the server program to transmit information and/or content (e.g., data, images, a user interface) to one or more computing devices 1250, receive information and/or content from one or more computing devices 1250, receive instructions from one or more devices (e.g., a personal computer, a laptop computer, a tablet computer, a smartphone), and so on.

In some configurations, the server 1252 is configured to perform the methods described in the present disclosure. For example, the processor 1312 and memory 1320 can be configured to perform the methods described herein.

In some configurations, data source 1202 can include a processor 1322, one or more data acquisition systems 1324, one or more communications systems 1326, and/or memory 1328. In some configurations, processor 1322 can be any suitable hardware processor or combination of processors, such as a CPU, a GPU, and so on. In some configurations, the one or more data acquisition systems 1324 are generally configured to acquire data, images, or both, and can include a patient monitor, such as a heart rate monitor, blood pressure device or catheter, ECG device, PPG device, and so forth. Additionally or alternatively, in some configurations, the one or more data acquisition systems 1324 can include any suitable hardware, firmware, and/or software for coupling to and/or controlling operations of a patient monitor system. In some configurations, one or more portions of the data acquisition system(s) 1324 can be removable and/or replaceable.

Note that, although not shown, data source 1202 can include any suitable inputs and/or outputs. For example, data source 1202 can include input devices and/or sensors that can be used to receive user input, such as a keyboard, a mouse, a touchscreen, a microphone, a trackpad, a trackball, and so on. As another example, data source 1202 can include any suitable display devices, such as an LCD screen, an LED display, an OLED display, an electrophoretic display, a computer monitor, a touchscreen, a television, etc., one or more speakers, and so on.

In some configurations, communications systems 1326 can include any suitable hardware, firmware, and/or software for communicating information to computing device 1250 (and, in some configurations, over communication network 1254 and/or any other suitable communication networks). For example, communications systems 1326 can include one or more transceivers, one or more communication chips and/or chip sets, and so on. In a more particular example, communications systems 1326 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some configurations, memory 1328 can include any suitable storage device or devices that can be used to store instructions, values, data, or the like, that can be used, for example, by processor 1322 to control the one or more data acquisition systems 1324, and/or receive data from the one or more data acquisition systems 1324; to process data; present content (e.g., data, images, a user interface) using a display; communicate with one or more computing devices 1250; and so on. Memory 1328 can include any suitable volatile memory, non-volatile memory, storage, or any suitable combination thereof. For example, memory 1328 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 1328 can have encoded thereon, or otherwise stored therein, a program for controlling operation of patient monitor data source 1202. In such configurations, processor 1322 can execute at least a portion of the program to generate or measure data, transmit information and/or content (e.g., data, images, a user interface) to one or more computing devices 1250, receive information and/or content from one or more computing devices 1250, receive instructions from one or more devices (e.g., a personal computer, a laptop computer, a tablet computer, a smartphone, etc.), and so on.

In some configurations, any suitable computer-readable media can be used for storing instructions for performing the functions and/or processes described herein. For example, in some configurations, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

1. A method for determining critical closing pressure (Pcrit) of a patient, the method comprising the steps of:
accessing physiological patient data that characterizes pressure parameters and surrogate flow data, the physiological patient data characterizing at least two time points;
using a processor to determine Pcrit based on the physiological patient data; and
generating a report based on the determined Pcrit of the patient.

2. The method of claim 1, wherein the physiological patient data comprises blood pressure data corresponding to the at least two time points; and wherein using the processor to determine Pcrit comprises the steps of:
determining a pressure parameter corresponding to the at least two time points based on the blood pressure data;
determining flow surrogate data corresponding to the at least two time points based on the blood pressure data;
characterizing a pressure-flow relationship between the pressure parameter and flow surrogate data corresponding to the at least two time points; and
determining Pcrit as a pressure at a zero-flow condition based on the relationship between the pressure parameter and flow surrogate data.

3. The method of claim 2, wherein the pressure parameter is determined from the blood pressure data and is one of mean arterial pressure (MAP), systolic blood pressure (SBP), or diastolic blood pressure (DBP).

4. The method of claim 2, wherein the processor is further configured to determine pulse pressure (PP) and heart rate (HR) from the blood pressure data, and wherein the surrogate flow data is determined as PP*HR.

5. The method of claim 4, wherein:
the pressure parameter is determined from the blood pressure data and is one of MAP, SBP, or DBP;
characterizing a pressure-flow relationship comprises determining a linear fit of the PP*HR and pressure parameter corresponding to the at least two time points; and
determining Pcrit as a pressure at the zero-flow condition comprises determining the pressure parameter intercept of the linear fit.

6. The method of claim 1, wherein the physiological patient data comprises a blood pressure waveform measured during a plurality of cardiac cycles; and
wherein using the processor to determine Pcrit comprises the steps of:
accessing a machine learning algorithm, the machine learning algorithm being trained on paired blood pressure waveform data and Pcrit values for a plurality of subjects; and
applying the machine learning algorithm to determine Pcrit based on the blood pressure waveform.

7. The method of claim 1, wherein the physiological patient data comprises at least one of photoplethysmography (PPG) data, electrocardiogram (ECG) data, echocardiogram data, Doppler flow data, optical spectroscopy data, arterial tonometry data, ultrasound imaging data, or cardiovascular imaging data; and
wherein using the processor to determine Pcrit comprises the steps of:
accessing a machine learning algorithm, the machine learning algorithm being trained on paired physiological patient data and Pcrit values for a plurality of subjects; and
applying the machine learning algorithm to determine Pcrit based on the at least one of PPG data, ECG data, echocardiogram data, Doppler flow data, optical spectroscopy data, arterial tonometry data, ultrasound imaging data, or cardiovascular imaging data.

8. The method of claim 1, wherein the physiological patient data comprises at least one of PPG data, ECG data, echocardiogram data, Doppler flow data, optical spectroscopy data, arterial tonometry data, ultrasound imaging data, or cardiovascular imaging data; and
wherein using the processor to determine Pcrit comprises the steps of:
calibrating the at least one of PPG data, ECG data, echocardiogram data, Doppler flow data, optical spectroscopy data, arterial tonometry data, ultrasound imaging data, or cardiovascular imaging data to a blood pressure waveform;
determining a pressure parameter corresponding to the at least two time points based on the blood pressure waveform;
determining flow surrogate data corresponding to the at least two time points based on the blood pressure waveform;
characterizing a pressure-flow relationship between the pressure parameter and flow surrogate data corresponding to the at least two time points; and
determining Pcrit as a pressure at a zero-flow condition based on the relationship between the pressure parameter and flow surrogate data.

9. The method of claim 1, wherein the physiological patient data characterizing each of the at least two time points characterizes a distinct flow state based on natural variability of flow over time.

10. A system for measuring critical closing pressure (Pcrit) of a patient, the system comprising:
a pressure/flow measurement module configured to measure physiological patient data that characterizes pressure parameters and surrogate flow data at two or more time points;
a processor configured to:
identify pressure parameters characterizing the two or more time points based on the physiological patient data, the pressure parameters comprising at least one of MAP, SBP, or DBP;
identify surrogate flow data characterizing the two or more time points based on the physiological patient data;
map a pressure-flow relationship for the two or more time points and calculate a measure of Pcrit as a zero-flow intercept of the pressure-flow relationship; and
generate a report based on the measure of Pcrit of the patient.

11. The system of claim 10, wherein the physiological patient data comprises blood pressure data; the pressure parameter is MAP; the surrogate flow data is determined as PP*HR; and the pressure-flow relationship is MAP vs. PP*HR.

12. The system of claim 10, wherein the pressure/flow measurement module comprises a blood pressure monitor, and the physiological patient data includes blood pressure measurements of the two of more cardiac cycles.

13. The system of claim 12, wherein the blood pressure monitor is a pressure catheter placed in an artery of the patient, and the blood pressure measurements include a blood pressure waveform of the two of more cardiac cycles.

14. The system of claim 12, wherein the blood pressure monitor is a non-invasive blood pressure monitor.

15. The system of claim 10, further comprising a treatment module configured to provide a treatment to the patient, and wherein the processor is further configured to adjust a treatment parameter of the treatment based on the measure of Pcrit.

16. The system of claim 10, further comprising a user interface configured to alert a user based on the measure of Pcrit.

17. The system of claim 10, wherein the processor is further configured to calculate a measure of tissue perfusion pressure (TPP) as MAP-Pcrit.

18. The system of claim 10, wherein pressure/flow measurement module comprises a photoplethysmography (PPG) sensor and the patient data comprises a photoplethysmogram.

19. The system of claim 18, wherein the processor is further configured to determine a blood pressure measurement based on the patient data.

20. The system of claim 10, wherein the pressure/flow measurement module comprises at least one of an electrocardiogram (ECG) device, an echocardiogram device, an ultrasound imaging device, a cardiovascular imaging device, an optical spectroscopy device, an arterial tonometry device, or a Doppler flow measurement device, and the patient data comprises at least one of an ECG data, optical spectroscopy data, ultrasound images, cardiovascular images, or flow measurements.

21. The system of claim 10, further comprising a flow modulation module configured to modify a blood flow of the patient between the two or more time points.

22. A system for measuring critical closing pressure (Pcrit) of a patient, the system comprising:
a pressure/flow measurement module configured to determine physiological patient data that characterizes cardiovascular dynamics of two or more cardiac cycles;
a processor configured to:
access a trained machine learning algorithm; and
apply the trained machine learning algorithm to the physiological patient data to estimate a measure of Pcrit for the patient, wherein the trained machine learning algorithm was trained on training data comprising paired physiological patient data and Pcrit label data for a plurality of subjects.

23. The system of claim 22, wherein the physiological patient data comprises a measure of systolic blood pressure and diastolic blood pressure during the two or more cardiac cycles.

24. The system of claim 22, wherein the physiological patient data comprises at least one of PPG data, ECG data, optical spectroscopy data, arterial tonometry data, Doppler flow data, ultrasound images, or cardiovascular images.

25. The system of claim 22, wherein the training data further comprises blood pressure waveform data paired to the physiological patient data and Pcrit label data for the plurality of subjects.

26. The system of claim 22 wherein the physiological patient data further comprises a measure of blood pressure during at least one cardiac cycle.
